(19) United States
(12) Patent Application Publication
(10) Pub. No.: US 2011/0195030 A1
(43) Pub. Date: Aug. 11, 2011

(54) NANOPARTICLE COMPOSITIONS COMPRISING LIQUID OIL CORES

(75) Inventors: Russell J. Mumper, Chapel Hill, NC (US); Xiaowei Dong, Parsippany, NJ (US)

(73) Assignees: The University of North Carolina at Chapel Hill, Chapel Hill, NC (US); The University of Kentucky, Lexington, KY (US)

(21) Appl. No.: 13/122,996

(22) PCT Filed: Oct. 14, 2009
(86) PCT No.: PCT/US2009/060593

§ 371 Date: Apr. 7, 2011

Related U.S. Application Data
(60) Provisional application No. 61/105,691, filed on Oct. 15, 2008.

(51) Int. Cl. A61K 47/14; A61K 31/337; A61K 9/51; A61K 49/18; A61P 35/00; B01J 13/02; B82Y 40/00

(52) U.S. Cl. 424/9.32; 514/449; 424/490; 424/9.1; 424/9.3; 424/9.36; 514/786; 264/4.1; 977/883

(57) ABSTRACT

Nanocapsule and nanoemulsion particle compositions having improved physical and pharmacological properties are provided. The nanocapsule or nanoemulsion particle composition can comprise a pharmaceutically acceptable liquid oil phase, a surfactant, and optionally a co-surfactant. The liquid oil phase can comprise a monoglyceride, a diglyceride, a triglyceride, a propylene glycol ester, or a propylene glycol diester. In certain embodiments, the nanocapsule or nanoemulsion particle composition can be lyophilized and subsequently re-hydrated without increasing the mean particle size and/or adversely affecting the potency or efficacy of a therapeutic agent (e.g., paclitaxel) present in the nanocapsules or nanoemulsion particles.

### GOVERNMENT INTEREST

This invention was made with government support under NIH-NCI R01 CA1 15197 awarded by the National Institutes of Health. The government has certain rights in the invention.

### FIELD OF THE INVENTION

The present invention relates generally to the fields of medicine and pharmaceutics. More particularly, it relates to nanoemulsions, nanoemulsion particles, and nanocapsules and methods for making and using the same.

### BACKGROUND OF THE INVENTION

Limited options presently exist for the administration of certain therapeutic agents that have limited solubility in water. For example, paclitaxel is a very effective chemotherapeutic agent, but its utility is hindered by its lipophilicity and currently available formulations. One currently available formulation marketed under the trademark TAXOL comprises paclitaxel in a 50:50 (v/v) mixture of CREMOPHOR EL (polyethoxylated castor oil) and dehydrated alcohol. Serious side effects, such as hypersensitivity reactions, attributable to CREMOPHOR EL have been reported (Weiss et al., 1990). In clinical therapy, high doses of anti-histamines and glucocorticoids are co-administered with TAXOL to manage these adverse effects, but this strategy has raised the possibility of additional pharmacokinetic and pharmacodynamic issues with paclitaxel. To eliminate CREMOPHOR EL from the paclitaxel formulation, several alternative CREMOPHOR EL-free formulations of paclitaxel have been investigated. ABRAXANE is a CREMOPHOR EL-free paclitaxel formulation and was registered with the Food and Drug Administration (FDA) in 2005. Despite its improved clinical profile, ABRAXANE has generally not replaced TAXOL in cancer chemotherapy, mostly due to its high cost. Therefore, alternative and cost-effective parenteral formulations of paclitaxel are still needed.

Improved formulations are needed for many types of poorly-water soluble and insoluble drugs. It typically is difficult or not possible to freeze-dry colloidal suspensions even in the presence of cryoprotectants without substantial disruption of the colloidal suspensions. To the inventors' knowledge, the successful lyophilization of colloidal suspensions without the use of a cryoprotectant that protects the nanoparticles from the stresses of the freezing and thawing process has not been previously performed.

Further, the lyophilization of nanoparticles (NP), nanoemulsions or nanocapsules is thought to be even more challenging due to the existence of a very thin and fragile lipid envelope that might not withstand the mechanical stress of freezing. Even in the presence of one or more cryoprotectants, increases of particle size are likely to occur. Thus, a need exists for improved nanoemulsions, nanoemulsion particles, and nanocapsule formulations.

### SUMMARY OF THE INVENTION

The present invention overcomes limitations in the related art by providing nanoparticles, e.g., nanoemulsion particles and nanocapsules, having improved physical characteristics and stability. For example, as described in further detail below, nanoparticles were successfully lyophilized and re-hydrated without the addition of a cryoprotectant and without adversely affecting the particle size or function of the particles. Surprisingly, as shown in the below examples, instead of increasing particle size as might be expected, particle sizes were slightly reduced after lyophilization and re-hydration with a complete retention of the in vitro release properties and cytotoxicity profile.

The nano-based formulations of the present invention preferably comprise liquid oil cores. Various nanoparticle compositions in some embodiments of the present invention can comprise one or more of the following: a caprylic/capric triglyceride (e.g., MIGLYOL 812 and equivalents), a polyoxyethylene 20-stearyl ether (e.g., BRIJ 78 and equivalents) and/or d-alpha-tocopheryl polyethylene glycol 1000 succinate (e.g., vitamin E TPGS and equivalents). As would be appreciated by one of skill in the art, it is anticipated that modifications to the surfactants or liquid oil phase described in the below examples can be made without adversely affecting the resulting nanoparticle or nanoemulsion compositions.

In some embodiments, the various nanoemulsion, nanoemulsion particle, and nanocapsule compositions of the present invention can be made without heating, microfluidization, extrusion, high torque mixing, or high pressure mechanical agitation. In these embodiments, various thermosensitive agents (e.g., a therapeutic protein or peptide, and the like) can be included in the nanocapsules or nanoemulsion particles. In other embodiments, nanoemulsions, nanoemulsion particles, and nanocapsules of the present invention can be made using heating and stirring, without any need for high pressure mechanical agitation or microfluidization.

In various embodiments, the nanoemulsion particles and nanocapsules of the present invention can be lyophilized and subsequently re-hydrated without an increase in particle size and/or without any reduction in the potency or efficacy of a therapeutic agent (e.g., paclitaxel) present in the nanoemulsion particles or nanocapsules. In certain embodiments, lyophilization and subsequent re-hydration of nanoemulsion particles and nanocapsules of the present invention can result in at least 50%, at least 60%, at least 65%, at least 70%, at least 75%, at least 80%, at least 85%, at least 90%, at least 95%, or substantially all nanoparticles having a diameter less than about 300 nm prior to lyophilization and subsequent to re-hydration. The mean or median diameter of the nanoparticles can preferably remain less than about 300 nm before lyophilization and after re-hydration. To the inventors' knowledge, nanocapsules or nanoemulsion particles that can be lyophilized and subsequently re-hydrated without an increase in particle size or disruption of the therapeutic efficacy of a compound contained within the nanoparticles have not previously been described.

A first aspect of the present invention relates to a nanocapsule or nanoemulsion particle comprising a pharmaceutically acceptable liquid oil phase, a surfactant, and optionally a co-surfactant; wherein the liquid oil phase comprises one or more compounds having the structure:

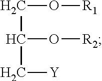

wherein:

Y is selected from the group consisting of H and —O—R₃;

R₁, R₂, and R₃ are each independently selected from the group consisting of

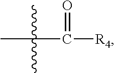

and H; wherein if R₁ is H and R₂ is H, then Y is not H and R₃ is not H;

R₄ is selected from the group consisting of C₁-C₂₅ alkyl, C₁-C₂₅ alkenyl, C₁-C₂₅ alkylyl, and

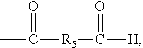

wherein R₅ is —(CH₂)ₓ—, wherein x is an integer from 1 to 12.

In certain embodiments, R₄ is selected from the group consisting of C₄-C₁₈ alkyl, C₈-C₂₅ alkenyl, and C₈-C₂₅ alkylyl. In certain embodiments, R₄ is —(CH₂)_{y}—, wherein y is an integer from 8 to 10.

In certain embodiments, the liquid oil phase comprises an esterified caprylic fatty acid, an esterified capric fatty acid, an esterified glycerin, or an esterified propylene glycol. The liquid oil phase can comprise a caprylic triglyceride, a capric or capric acid triglyceride, a linoleic triglyceride, a succinic triglyceride, a propylene glycol dicaprylate, or a propylene glycol dicaprate. The liquid oil phase can comprise a compound selected from the group consisting of triglyceryl monoleate, glyceryl monostearate, a medium chain monoglyceride or diglyceride, glyceryl monocaprate, glyceryl monocaprylate, decaglycerol decaoleate, triglycerol monooleate, triglycerol monostearate, a polyglycerol ester of a mixed fatty acid, hexaglycerol dioleate, a decaglycerol mono- or dioleate, propylene glycol dicaprate, propylene glycol dicaprylate/dicaprate, glyceryl tricaprylate/caprate, glyceryl tricaprylate/caprate/laurate, glyceryl tricaprylate/caprate, triacetin, propylene glycol di-(2-ethylhexanoate), glyceryl tricaprylate/caprate/linoleate, glyceryl tricaprate, glyceryl tricaprylate, and glyceryl triundecanoate.

In various embodiments, the liquid oil phase can comprises a naturally derived liquid oil, such as corn oil, coconut oil, sunflowerseed oil, vegetable oil, cottonseed oil, mineral oil, peanut oil, sesame oil, soybean oil, or olive oil.

In some embodiments, the liquid oil phase comprises a caprylic/capric triglyceride, such as MIGLYOL 810 or MIGLYOL 812; a caprylic/capric/linoleic triglyceride, such as MIGLYOL 818; a caprylic/capric/succinic triglyceride, such as MIGLYOL 829; or a propylene glycol dicaprylate/dicaprate, such as MIGLYOL 840. In some embodiments, the liquid oil phase comprises a caprylic/capric triglyceride, such as MIGLYOL 810 or MIGLYOL 812. In other embodiments, the liquid oil phase comprises a glyceryl trihexanoate, such as MIGLYOL 612.

The surfactant or the co-surfactant can have a hydrophilic-lipophilic balance (HLB) of from about 6 to about 20, including 6, 7, 8, 9, 10, 11, 12, 13, 14, 15, 16, 17, 18, 19, and 20, or from about 8 to about 18, including 8, 9, 10, 11, 12, 13, 14, 15, 16, 17, and 18. In certain embodiments, the surfactant and the co-surfactant have a hydrophilic-lipophilic balance (HLB) of from about 8 to about 18, including 8, 9, 10, 11, 12, 13, 14, 15, 16, 17, and 18. The surfactant can be selected from the group consisting of a polyoxyethylene alkyl ether, a polyoxyethylene sorbitan fatty acid ester, a phospholipid, a polyoxyethylene stearate, a fatty alcohol, and hexadecyltrimethyl-ammonium bromide. The surfactant can be conjugated to polyethylene glycol, polyoxyethylene, a cell-targeting ligand, a small molecule, a peptide, a protein, or a carbohydrate. The surfactant can be d-alpha-tocopheryl polyethylene glycol 1000 succinate (TPGS) or polyoxyethylene 20-stearyl ether. In certain embodiments, the surfactant is polyoxyethylene 20-stearyl ether, the co-surfactant is d-alpha-tocopheryl polyethylene glycol 1000 succinate (TPGS). In some embodiments, the liquid oil phase comprises a caprylic/capric triglyceride, for example, MIGLYOL 810 or MIGLYOL 812; wherein the surfactant is d-alpha-tocopheryl polyethylene glycol 1000 succinate (TPGS); and wherein the co-surfactant is polyoxyethylene 20-stearyl ether.

In representative embodiments, the nanocapsules or nanoemulsion particles can be produced by admixing about 2.5 mg of MIGLYOL 812, about 1.5 mg of TPGS, and about 3.5 mg of polyoxyethylene 20-stearyl ether, per 1 mL aqueous solution. The nanocapsules or nanoemulsion particles can comprise a ratio of liquid oil phase:TPGS:polyoxyethylene 20-stearyl ether of about 1-3:1-3:1-5 (w:w:w). In particular embodiments, the nanocapsules or nanoemulsion particles further comprise paclitaxel.

In certain embodiments, the nanocapsules or nanoemulsion particles further comprise a therapeutic agent, such as a substantially water-insoluble or a lipophilic drug. The therapeutic agent can be selected from the group consisting of a small molecule, a chemotherapeutic agent, an anti-viral agent, a bacteriostatic or anti-bacterial agent, and an anti-fungal agent. The entrapment efficiency of the therapeutic agent can be at least 50%, at least 80%, or at least 90% in the nanocapsules or nanoemulsion particles. The therapeutic agent can be a chemotherapeutic agent, such as paclitaxel. The nanocapsules or nanoemulsion particles can be lyophilized and subsequently rehydrated without substantially affecting the potency of the composition after re-hydration, as compared to the potency of the composition prior to the lyophilization. In certain embodiments, the therapeutic agent is a chemotherapeutic agent, and the potency includes the in vitro cytotoxicity of the nanocapsules or nanoemulsion particles. The therapeutic agent can be present in the nanocapsules or nanoemulsion particles at a weight ratio of at least 6% of the liquid oil phase. The nanocapsules or nanoemulsion particles can or can not comprise a cryoprotectant. The nanocapsules or nanoemulsion particles can or can not have been lyophilized, or they can be present in a substantially aqueous solution. In certain embodiments, the nanocapsules or nanoemulsion particles have been rehydrated or re-suspended from a previously lyophilized composition.

The nanocapsules or nanoemulsion particles can be designed via a method comprising Taguchi array and sequential simplex optimization. Substantially all of the nanocapsules or nanoemulsion particles can have particle size diameters less than about 300 nm. The composition can be free or essentially free of polyethoxylated castor oil. The composition can be formulated for parenteral administration (e.g., intramuscular, subcutaneous, intraperitoneal, intratumoral, or intravenous administration). In other embodiments, the composition can be formulated for topical, rectal, oral, inhalation, intranasal, transdermal, or buccal administration. The composition can be further defined as a pharmaceutically acceptable formulation, wherein the formulation is free or essentially free of viable bacteria and viruses. The presently disclosed compositions also can be used for the preparation of a medicament for use in treating a disease, condition, or affliction.

Another aspect of the present invention relates to a method of treating a disease comprising administering the composition of the present invention to a subject in need of such treatment, wherein the nanocapsules or nanoemulsion particles comprise at least one bioactive agent, wherein at least one bioactive agent has a therapeutic or a prophylactic activity for the disease. The bioactive agent can be selected from the group consisting of a small molecule, a therapeutic agent, including a chemotherapeutic agent, an anti-viral agent, a bacteriostatic or anti-bacterial agent, and an anti-fungal agent. The therapeutic agent can be substantially water insoluble or lipophilic. The disease can be selected from the group consisting of a hyperproliferative disease, a cancer, or an inflammatory disease. In certain embodiments, the disease is cancer, and wherein the therapeutic agent is an anti-cancer agent. The anti-cancer agent can be a chemotherapeutic agent (e.g., paclitaxel, docetaxel, etoposide, or 7-ethyl-10-hydroxy-camptothecin (SN-38)). The chemotherapeutic agent can be substantially water-insoluble or lipophilic. In certain embodiments, the method is further defined as a method of overcoming resistance to the anti-cancer agent. The administration can comprise parenteral administration (e.g., intramuscular, subcutaneous, intraperitoneal, intratumoral, or intravenous administration).

Yet another aspect of the present invention relates to a method of making a composition of the present invention, comprising admixing the liquid oil phase, the surfactant, and the co-surfactant with an aqueous solvent or a non-aqueous solvent; wherein high pressure mechanical agitation, microfluidization, or heating is not required to produce the nanoparticles or nanocapsules. The method can comprise heating the liquid oil phase, the surfactant, and the co-surfactant with the aqueous solvent or the non-aqueous solvent during the admixing to produce the nanoparticles or the nanocapsules. In other embodiments, the liquid oil phase, the surfactant, and the co-surfactant are not heated during the admixing with the aqueous solvent or the non-aqueous solvent. The method can further comprise adding a solvent to the liquid oil phase, the surfactant, and the co-surfactant, prior to admixing with the aqueous solvent, e.g., water, wherein the solvent is selected from the group consisting of ethanol, acetone, or ethyl acetate. The method can further comprise admixing a therapeutic agent with the liquid oil phase, the surfactant, and the co-surfactant. In some embodiments, the therapeutic agent can be a thermosensitive compound, such as, e.g., a protein, a peptide, or a nucleic acid.

Certain aspects of the presently disclosed subject matter having been stated hereinabove, which are addressed in whole or in part by the presently disclosed subject matter, other objects, features and advantages of the present invention will become evident as the description proceeds when taken in connection with the accompanying Examples and Drawings as best described herein below. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention can be better understood by reference to one or more of these drawings in combination with the detailed description of specific embodiments presented herein. Having thus described the presently disclosed subject matter in general terms, reference will now be made to the accompanying drawings, which are not necessarily drawn to scale, and wherein:

FIG. 1: The principles of sequential simplex optimization for two variables using variable-size simplex rules on the response surface (Walters et al., 1991). The starting simplex consists of vertexes 1, 2 and 3, where 1 gives the worst response. The second simplex consists of vertexes 2, 3, and 4 after a reflection and expansion. Finally, the movement of the simplex results in the simplex 12, 14, and 15, which indicates the optimum.

FIG. 2: Particle size of BTM nanoparticles before and after lyophilization (and rehydration). Six different batches were tested for both blank BTM nanoparticles and paclitaxel (PX)-loaded BTM nanoparticles. For all tested NP formulations, P.I. values ranged from 0.03 to 0.35 indicating uniform, mono-dispersed NPs. Data are presented as the mean particle size of three separate measurement of each batch.

FIG. 3: Long-term stability of paclitaxel nanoparticles stored at 4° C. Three different batches of PX-loaded BTM and G78 nanoparticles were monitored for particle sizes over five months. For all tested samples, P.I.<0.35. Data are presented as the mean particle size of three separate measurement of each batch.

FIG. 4: Stability of paclitaxel nanoparticles in PBS at 37° C. PX BTM nanoparticles, reconstituted lyophilized PX BTM nanoparticles and PX G78 nanoparticles were monitored for particle sizes for 102 h. For all tested samples, P.I.<0.35. Data are presented as the mean particle size of three separate measurements of each batch.

FIG. 5: Differential scanning calorimetry (DSC) for G78 nanoparticles. (A) DSC analysis of nanoparticles was performed immediately after concentrating nanoparticles (“dry”). (B) The concentrated nanoparticles were dried by desiccations for two days prior to DSC analysis (“wet”). GT means glyceryl tridodecanoate.

FIG. 6: Release of PX from PX nanoparticles at 37° C. Paclitaxel release was measured using the dialysis method in PBS (pH 7.4) with 0.1% Tween 80 as described in the Method section. Data are presented as the mean±SD (n=4).

FIG. 7: Uptake of calcein AM over 1 h after defined exposure of samples in NCI/ADR-RES cells. Concentration of blank BTM nanocapsules was calculated based on paclitaxel equivalent dose. Each sample was measured in triplicate.

FIG. 8: Dose response of blank BTM nanocapsules in calcein AM assay in NCI/ADR-RES cells. Concentrations of blank BTM nanocapsules were calculated based on paclitaxel equivalent doses. Each sample was measured in triplicate.

FIG. 9: Blank BTM nanocapsules deplete ATP in P-glycoprotein (P-gp) overexpressing NCI cells, but not in non P-gp-overexpressing MDA-MB-468 cells.

FIG. 10: Freeze-fracture TEM and SEM of blank BTM nanoparticles.

FIG. 11: In-vivo anticancer efficacy study #1 using pegylated PX BTM NPs in resistant mouse NCI/ADR-RES xenografts. On Day (−7), 18-19 g female nude mice received 4×10⁶ cells by s.c. injection. Mice (n=4/group) were dosed i.v. with PX (4.5 or 2.25 mg/kg) by tail vein injection on day 0 and 7. The corresponding nanoparticle dose was 210 or 105 mg NPs/kg, respectively. Data are presented as the mean±SD.

FIG. 12: In-vivo anticancer efficacy study #2 using pegylated PX BTM NPs in resistant mouse NCI/ADR-RES xenografts. Female nude mice received 4×10⁶ cells by s.c. injection. Mice (n=6/group) were dosed i.v. with PX (4.5 mg/kg) by tail vein injection on day 0, 7, 14, and 21 in the form of either TAXOL, PX BTM NPs, or TAXOL spiked in blank BTM NPs. TAXOL (20 mg/kg) near or at the maximum tolerated dose as well as blank NPs with a dose of NPs equal to that of PX BTM NPs were added as controls. The corresponding nanoparticle dose was 210 mg NPs/kg, respectively. Data are presented as the mean±SD.

FIG. 13: Retreatment of selected groups in study #2 (shown in FIG. 12). Left Panel: TAXOL-failed mice from efficacy study #2 were combined and then treated with PX BTM NPs to determine if the NPs could salvage the TAXOL-failed mice. Doses and dosing schedule of PX BTM NPs to the TAXOL-failed mice is shown in the legend. As depicted in the figure, the treatment of TAXOL-failed mice with PX BTM NPs significantly (p<0.05) reduced tumor sizes demonstrating efficacy in treating TAXOL-failed mice. Right Panel: Previously PX BTM NP-treated mice were retreated with PX BTM NPs at the doses and dosing schedule shown in the legend. The retreatment significantly (p<0.05) reduced tumor sizes demonstrating that retreatment with PX BTM NPs provided efficacy. Data are presented as the mean±SD.

FIG. 14: BTM NPs were prepared with accessible diethylenetriaminepentaacetic acid (DTPA) on the surface of the NPs using methods described by Zhu et al., “Nanotemplate-engineered nanoparticles containing gadolinium for magnetic resonance imaging of tumors,” Invest Radiol. 43(2):129-40 (2008). The BTM-DTPA-Gd NPs were injected into nude mice bearing A549 tumors. Five hours after injection, MRI images were obtained using a 9.4T Micro-MRI. The results showed that the BTM-DTPA-Gd NPs provided positive tumor contrast (panel at right) were control (panel on left).

### DETAILED DESCRIPTION

The presently disclosed subject matter now will be described more fully hereinafter with reference to the accompanying Drawings, in which some, but not all embodiments of the presently disclosed subject matter are shown. Many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims. Although specific terms are employed herein, they are used in a generic and descriptive sense only and not for purposes of limitation.

Throughout this application, the term “about” is used to indicate that a value includes the inherent variation of error for the device, the method being employed to determine the value, or the variation that exists among the study subjects. Thus, the term “about,” when referring to a value is meant to encompass, but is not limited to, variations of, in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification can mean “one,” but it also is consistent with the meaning of “one or more,” “at least one,” and “one or more than one.” Thus, for example, reference to “a sample” includes a plurality of samples, unless the context clearly is to the contrary (e.g., a plurality of samples), and so forth.

It is contemplated that any embodiment discussed in this specification can be implemented with respect to any method or composition of the invention, and vice versa. Furthermore, compositions of the invention can be used to achieve methods of the invention.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended, i.e., are non-exclusive, and do not exclude additional, unrecited elements or method steps, except where the context requires otherwise.

### I. Nanoemulsions, Nanoemulsion Particles and Nanocapsules

The present invention provides nanoemulsions, nanoemulsion particles, and nanocapsules having improved physical and pharmacological properties. The nanocapsule or nanoemulsion particle compositions can comprise a pharmaceutically acceptable liquid oil phase, a surfactant, and optionally a co-surfactant, wherein the liquid oil phase comprises a monoglyceride, a diglyceride, a triglyceride, a propylene glycol monoester, a propylene glycol diester, or a mixture of two, three, four, or more different oils.

A “liquid oil phase,” as used herein, refers to an oil that is substantially liquid at room temperature (70-75° F.). Various liquid oil phases can be used with the present invention, as described herein. In certain embodiments, nanocapsules or nanoemulsion particles of the present invention can comprise a monoglyceride, a diglyceride, a triglyceride, or a monoester or diester of propylene glycol, or a mixture of two, three, four or more oils. In certain embodiments, in instances where a monoglyceride exhibits substantial hydrophilicity, it can be desirable to use the monoglyceride as a surfactant rather than a component of the liquid oil phase; in other embodiments, it can be desirable to include a substantially lipophilic monoglyceride in a liquid oil phase according to the present invention.

The terms “semi-solid” or “quasi solid” refers to a substance that has physical properties similar to a solid in some respects (e.g., an ability to support its own weight and substantially hold its shape), but a quasi-solid also shares some properties of liquids, such as shape conformity to something applying pressure to it, or the ability to flow under pressure. Quasi-solids also are known as amorphous solids because at the microscopic scale they are disordered, unlike traditional crystalline solids. While it is anticipated that the core of a nanoparticle can comprise a semi-solid or quasi solid compound, in certain embodiments nanoparticles of the present invention do not have semi-solid or quasi solid cores. In other embodiments, under the proper conditions (e.g., sufficient cooling, and the like) nanoparticles, nanoemulsions, and/or nanocapsules of the present invention can have substantially semi-solid or quasi solid cores.

In certain embodiments, the nanocapsule or nanoemulsion particle can be lyophilized and subsequently re-hydrated without increasing the mean particle size and/or adversely affecting the potency or efficacy of a therapeutic agent (e.g., paclitaxel) present in the nanocapsules or nanoemulsion particles. The nanocapsule or nanoemulsion particle of the present invention can comprise a substantially water-insoluble or lipophilic therapeutic agent, drug, imaging agent, for example, a magnetic resonance imaging (MRI) imaging agent, nucleic acid, protein, or peptide. Thermosensitive compounds also can be comprised in the nanoparticles and nanoemulsion particles of the present invention. In certain embodiments, the nanocapsules or nanoemulsion particles of the present invention can be used to overcome cancer resistance to a chemotherapeutic agent (e.g., resistance to paclitaxel by cancer cells). Certain nanocapsules or nanoemulsion particles of the present invention are stable at about 4° C. for at least five months or more.

More particularly, lipid-based particulate delivery systems, including liposomes, micelles, nanoemulsion particles and nanocapsules having a liquid core, and solid lipid nanoparticles have been developed to solubilize poorly water-soluble and lipophilic drugs. These lipid-based systems have the advantage of being comprised of bio-derived and/or biocompatible lipids that often result in lower toxicity. In general, the lipid-based systems are made from the combination of lipophilic (oil), amphiphilic (surfactant) and hydrophilic (water) excipients. Formulation approaches typically involve a highly interactive process of experimentally investigating many variables including type and amount of excipients, excipient combinations, and processes (i.e., high-pressure homogenization, microfluidization, extrusion, microemulsion precursors, and the like). Appropriate type and amount of excipients are critical variables, especially in the case of microemulsion precursors to prepare lipid-based systems. Typically, phase diagrams with the blends of different excipients are first developed using the water titration method. Then, combinations of excipients and the drug substance are further optimized for their phase behavior and thermodynamic stability (Kang et al., 2004; Bummer, 2004). However, when several surfactants and/or oils are used, construction of phase diagrams can be tedious, expensive, and time consuming. As a result and as described in further detail below, the combination of Taguchi array and/or sequential simplex optimization can be used to optimize nanoemulsion particles and nanocapsules of the present invention.

Preferably, the nanoemulsion particles and nanocapsules of the present invention comprise an oil phase, a surfactant, and optionally a co-surfactant. The presently disclosed nanoemulsion particles and nanocapsules comprise substantially liquid cores and thus differ from nanoparticles having solid cores. For example, U.S. Pat. No. 7,153,525 discloses nanoparticles having solid cores comprising “meltable” solid lipid excipients; in contrast to these solid nanoparticles and as shown in the below examples, nanoemulsion particles and nanocapsules of the present invention preferably have a liquid oil core. Further, certain nanoemulsion particles or nanocapsules of the present invention can be lyophilized without the use of a cryoprotectant, and can be used to overcome certain forms of chemotherapeutic resistance (e.g., paclitaxel resistance).

The term “nanoparticle,” as used herein, refers to particles that have diameters below one micrometer in diameter and include nanoemulsion particles and nanocapsules. “Stable nanoparticles” remain largely unaffected by environmental factors, such as temperature, pH, body fluids, or body tissues. The nanoparticles can contain, or have adsorbed to or be conjugated with, many different materials for various pharmaceutical and engineering applications including, but not limited to, plasmid DNA for gene therapy and genetic vaccines, peptides and proteins or small drug molecules, magnetic substances for use as nanomagnets, lubricants, or chemical, thermal, or biological sensors. The nanoparticles preferably have a diameter of less than about 300 nanometers and more preferably the nanoparticles have a diameter of less than about 200 nanometers.

As used herein, a “microemulsion” is a stable biphasic mixture of two immiscible liquids stabilized by a surfactant and usually a co-surfactant. Microemulsions are thermodynamically stable, isotropically clear, form spontaneously without excessive mixing, and have dispersed droplets in the range of about 5 nm to 140 nm. In contrast, emulsions are opaque mixtures of two immiscible liquids. Emulsions are thermodynamically unstable systems, and usually require the application of high-torque mechanical mixing or homogenization to produce dispersed droplets in the range of about 0.2 to 25 μm. Both microemulsions and emulsions can be made as water-in-oil or oil-in-water systems. Whether water-in-oil or oil-in-water systems will form is largely influenced by the properties of the surfactant. The use of surfactants that have hydrophilic-lipophilic balances (HLB) of about 3-6, including 3, 4, 5, 6 and fractions thereof, tend to promote the formation of water-in-oil microemulsions, while those with HLB values of about 8-18, including 8, 9, 10, 11, 12, 13, 14, 15, 16, 17, 18, and fractions thereof, tend to promote the formation of oil-in-water microemulsions.

Microemulsions were first described by Hoar and Schulman in 1943 after they observed that a medium chain alcohol could be added to an emulsion to produce a clear system within a defined “window,” now referred to as a microemulsion window. A unique physical aspect of microemulsions is the very low interfacial surface tension (γ) between the dispersed and continuous phases. In a microemulsion, the small size of the dispersed droplets presents a very large interface. A thermodynamically stable microemulsion can only be made if the interfacial surface tension is low enough so that the positive interfacial energy (γA, where A equals the interfacial area) can be balanced by the negative free energy of mixing (δGₘ). The limiting γ value needed to produce a stable microemulsion with a dispersed droplet of 10 nm, for example, can be calculated as follows: δGₘ=−TδSₘ (where T is the temperature and the entropy of mixing δSₘ, is of the order of the Boltzman constant k_{B}). Thus, k_{B}T=4πr²γ and the limiting γ value is calculated to be k_{B}T/4πr² or 0.03 mN m⁻¹. Often, a co-surfactant is required in addition to the surfactant to achieve this limiting interfacial surface tension.

In addition to their unique properties as mentioned above, microemulsions have several advantages for use as delivery systems for pharmaceutical products, including: i) increased solubility and stability of drugs incorporated into the dispersed phase; ii) increased absorption of drugs across biological membranes; iii) ease and economy of scale-up (since expensive mixing equipment is often not needed); and iv) rapid assessment of the physical stability of the microemulsion (due to the inherent clarity of the system). For example, oil-in-water microemulsions have been used to increase the solubility of lipophilic drugs into formulations that are primarily aqueous-based (Constantinides, 1995). Both oil-in-water and water-in-oil microemulsions also have been shown to enhance the oral bioavailability of drugs, including peptides (Bhargava et al., 1987; Constantinides, 1995).

Although microemulsions have many potential advantages they also have potential limitations, including: a) they are complex systems and often require more development time; b) a large number of the proposed surfactants/co-surfactants are not pharmaceutically acceptable (Constantinides, 1995); and c) the microemulsions are not stable in biological fluids due to phase inversion. Thus, the microemulsions themselves are not effective in delivering drugs intracellularly or targeting drugs to different cells in the body. Further, the development of a microemulsion involves the very careful selection and titration of the dispersed phase, the continuous phase, the surfactant and the co-surfactant. Time consuming pseudo-phase ternary diagrams involving the preparation of a large number of samples must be generated to find the existence of the “microemulsion window,” if any (Attwood, 1994). In general, a water-in-oil microemulsion typically is much easier to prepare than an oil-in-water microemulsion. The former system is useful for formulating water-soluble peptides and proteins to increase their stability and absorption while the latter system is preferred for formulating drugs with little or no aqueous solubility.

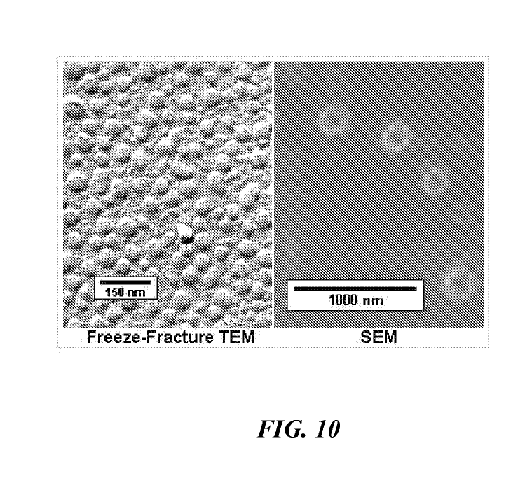

A nanoemulsion is defined as a mixture of two immiscible liquids. With nanoemulsions, an inner phase can act as an emulsifier, resulting in nanoemulsion where the inner state disperses into nano-sized droplets within the outer phase. Nanoemulsion particles can exist as water-in-oil and oil-in-water forms, where the core of the particle is either water or oil, respectively. Nanoemulsions can be thermodynamically stable particles characterized by having a very low surface tension that produces a very large surface area (Sarker, 2005; Anton et al., 2008). Nanoemulsions and nanocapsules can thus certain significant advantages (Anton et al., 2008). Nanocapsules are similar to a nanoemulsion except that the nanocapsule can have a thin solid shell or wall encasing the liquid dispersed phase. See, for example, FIG. 10, right panel.

In the present invention, the nanoemulsions or nanocapsules are sometimes referenced to as a nanoparticle. Nanoemulsion particles and nanocapsules suitable for use with the presently disclosed subject matter have particle sizes less than 300 nm, preferably less than 200 nm. Generally, a nanoparticle, a nanoemulsion particle or a nanocapsule refer to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm). In some embodiments, the nanoemulsion particle or nanocapsule is a spherical particle, or substantially spherical particle, having a core, e.g., a liquid core, diameter between about 2 nm and about 300 nm (including about 2, 5, 10, 20, 50, 60, 70, 80, 90, 100, 200, and 300 nm). In some embodiments, the nanoemulsion particle or nanocapsule has a core diameter between about 2 nm and about 200 nm (including about 2, 5, 10, 20, 30, 40, 50, 60, 70, 80, 90, 100, 110, 120, 130, 140, 150, 160, 170, 180, 190, and 200 nm). In some embodiments, the nanoemulsion particle or nanocapsule has a core diameter between about 2 nm and about 100 nm (including about 2, 5, 10, 20, 30, 40, 50, 60, 70, 80, 90, and 100 nm) and in some embodiments, between about 20 nm and 100 nm (including about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nm).

Nanoparticles can be measured by a conventional technique, such as photon correlation spectroscopy or other light scattering techniques or electron microscopy with measured particles in the nano-size range. Nanoparticles of the present invention can exhibit improved drug loading, drug release rates, drug pharmacokinetics, biodistribution, and/or reduced toxicities associated with the administration of a therapeutic agent.

### A. Pharmaceutically Acceptable Oil-Phases

It is envisioned that various oil phases can be used to prepare the nanocapsules or nanoemulsion particles of the present invention. Liquid oil phases are known to those skilled in the art. The primary criteria for suitable liquid oil phases are that the oil is (1) a liquid; (2) either immiscible with water, insoluble in water, or poorly-water soluble; and (3) biocompatible. In various embodiments, a liquid oil phase of the present invention can comprise one or more compounds of the structure:

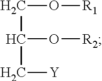

wherein:

Y is selected from the group consisting of H and —O—R₃;

R₁, R₂, and R₃ are each independently selected from the group consisting of

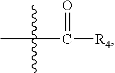

and H; wherein if R₁ is H and R₂ is H, then Y is not H and R₃ is not H;

R₄ is selected from the group consisting of C₁-C₂₅ substituted or unsubstituted alkyl, C₁-C₂₅ substituted or unsubstituted alkenyl, C₁-C₂₅ substituted or unsubstituted alkylyl, and

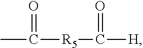

wherein R₅ is —(CH₂)ₓ—, wherein x is an integer from 1 to 12.

Preferably, R₄ is selected from the group consisting of C₁-C₂₅ alkyl, C₁-C₂₅ alkenyl, and C₁-C₂₅ alkylyl, and

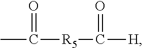

wherein R₅ is —(CH₂)ₓ—, wherein x is an integer from 1 to 12.

In the above structure, it is important to note that if one or more of R₁, R₂, and/or R₃ are

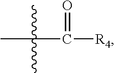

then a different R₄ group can be associated with R₁, R₂, and/or R₃ (that is, R₁, R₂, and/or R₃ do not need to have the same R₄ group).

In certain embodiments, R₁ or R₂ is

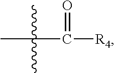

wherein R₄ is selected from the group consisting of C₄-C₁₈ alkyl, C₈-C₂₅ alkenyl, and C₈-C₂₅ alkylyl. In further embodiments, R₄ is —(CH₂)_{y}—, wherein y is an integer from 8 to 10. In certain embodiments, R₁, R₂, and/or R₃ can be a caprylic (C₈₋) group, a capric (C₁₀₋) group, a linoleic group, or a succinic group.

It will be generally appreciated by one of skill in the art that propylene glycol and glycerol are water miscible and are generally not acceptable for use as the only component of an oil phase. Further, it will generally be appreciated that R₁, R₂, and Y are preferably sufficiently lipophilic to result in a compound that is immiscible with water.

As used herein the term “alkyl” generally refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butyryl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

More particularly, the term “alkyl” when used without the “substituted” modifier refers to a non-aromatic monovalent group, having a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃(Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

More particularly, the term “substituted alkyl” refers to a non-aromatic monovalent group, having a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, CH₂Br, —CH₂SH, —CF₃, —CH₂CN, —CH₂C(O)H, —CH₂C(O)OH, —CH₂C(O)OCH₃, CH₂C(O)NH₂, —CH₂C(O)NHCH₃, —CH₂C(O)CH₃, —CH₂OCH₃₅—CH₂OCH₂CF₃, CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkenyl” as used herein refers to a straight or branched hydrocarbon of a designed number of carbon atoms containing at least one carbon-carbon double bond. Examples of “alkenyl” include vinyl, allyl, 2-methyl-3-heptene, and the like. More particularly, the term “alkenyl” when used without the “substituted” modifier refers to a monovalent group, having a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, CH₂CH═CH₂ (allyl), —CH₂CH═CHCH₃, and —CH═CH—C₆H₅.

The term “substituted alkenyl” refers to a monovalent group, having a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and CH═CHBr, are non-limiting examples of substituted alkenyl groups.

The term “alkynyl” as used herein refers to a straight or branched hydrocarbon of a designed number of carbon atoms containing at least one carbon-carbon triple bond. Examples of “alkynyl” include propargyl, propyne, and 3-hexyne. More particularly, the term “alkynyl” when used without the “substituted” modifier refers to a monovalent group, having a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and CH₂C≡CCH₃, are non-limiting examples of alkynyl groups. The term “substituted alkynyl” refers to a monovalent group, having a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two a-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups C≡CCFH— and —C≡CHCH(Cl)— are non-limiting examples of substituted alkynediyl groups.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

As would be appreciated by one of skill in the art, various synthesis reactions and schemes can be used to produce a monoglyceride, diglyceride, triglyceride, ester of propylene glycol, or diester of propylene glycol. For example, an alcohol group present on a glycerol or propylene glycol backbone can be reacted with a carboxylic acid group present on, e.g., caprylic acid, capric acid, linoleic acid, or a dicarboxylic acid, such as malonic acid, succinic acid, glutaric acid, adipic acid, pimelic acid, suberic acid, azelaic acid, or sebacic acid. Carboxylic acids react readily with alcohols in the presence of catalytic amounts of mineral acids to yield esters (see, e.g., Streitwieser and Heathcock, 1985). Additional esterification methods also can be used to produce an oil phase to be used in nanocapsules or nanoemulsion particles of the present invention.

Certain nanoemulsion particles or nanocapsules of the present invention comprise a liquid oil phase comprising a MIGLYOL neutral oil (Sasol Germany GmbH, Witten, Germany). MIGLYOL neutral oils are esters of saturated coconut and palmkernel oil-derived caprylic and capric fatty acids and glycerin or propylene glycol. Some examples of useful MIGLYOLs include MIGLYOL 810 and 812 (Caprylic/Capric Triglyceride), MIGLYOL 818 (Caprylic/Capric/Linoleic Triglyceride), MIGLYOL 829 (Caprylic/Capric/Succinic Triglyceride), MIGLYOL 612 (Glyceryl Trihexanoate), and MIGLYOL 840 (Propylene Glycol Dicaprylate/Dicaprate). MIGLYOL neutral oils generally are free of additives, such as antioxidants, solvents, and catalyst residues, with the exception of MIGLYOL 818, which includes an antioxidant.

More particularly, MIGLYOL 810 and MIGLYOL 812 (CAS Registry No. 73398-61-5) are triglycerides of the fractionated plant fatty acids C₈ and C₁₀ and can alternatively be referred to as medium-chain triglycerides, fractionated coconut oil, and more generally, caprylic/capric triglyceride. MIGLYOL 810 and MIGLYOL 812 differ only in C₈/C₁₀ ratio. MIGLYOL 818 (CAS Registry No. 67701-28-4) is a glycerin ester of the fractionated plant fatty acids C₈ and C₁₀, and contains about 4-5% linoleic acid. MIGLYOL 829 (CAS Registry No. 91744-56-8) is a glycerin ester of the fractionated plant fatty acids C₈ and C₁₀, combined with succinic acid. MIGLYOL 840 (CAS Registry No. 68583-51-7) is a propylene glycol diester of saturated plant fatty acids with chain lengths of C₈ and C₁₀. The compositions of fatty acids in representative MIGLYOL neutral oils are provided in Table 1.

TABLE 1
Fatty Acid Compositions of Representative MIGLYOL Oils

| MIGLYOL | 612 | 810 | 812 | 818 | 829 | 840 |
|---|---|---|---|---|---|---|
| Caproic acid (C_{6:0}) | | max. 2% | max. 2% | max. 2% | max. 2% | max. 2% |
| Caprylic acid (C_{8:0}) | | 65-80% | 50-65% | 45-65% | 45-55% | 65-80% |
| Capric acid (C_{10:0}) | | 20-35% | 30-45% | 30-45% | 30-40% | 20-35% |
| Laurie acid (C_{12:0}) | | max. 2% | max. 2% | max. 3% | max. 3% | max. 2% |
| Myristic acid (C_{14:0}) | | max. 1% | max. 1% | max. 1% | max. 1% | max. 1% |
| Linoleic acid (C_{18:2}) | | | | 2-5% | | |
| Succinic acid | | | | | 15-20% | |
| Glyceryl Trihexanoate | 100% | | | | | |

One of ordinary skill in the art would recognize that MIGLYOL neutral oils are disclosed herein as exemplary embodiments and equivalent liquid oils from other sources are contemplated for use with the presently disclosed compositions and methods.

Other types of oil phases can be used with the present invention including monoglycerides, diglycerides, triglycerides, esters propylene glycol, and diesters or propylene glycol, which can comprise suitable lipophilic groups linked via an ester bond to the glycerol or propylene glycol backbone. Other oil phases that can be used with the present invention include, but are not limited to: triglyceryl monoleate, glyceryl monostearate, medium chain mono- and diglycerides, glyceryl monocaprate, glyceryl monocaprylate, decaglycerol decaoleate, triglycerol monooleate, triglycerol monostearate, polyglycerol ester of mixed fatty acids, hexaglycerol dioleate, decaglycerol mono- or dioleate, propylene glycol dicaprate, propylene glycol dicaprylate/dicaprate, glyceryl tricaprylate/caprate, glyceryl tricaprylate/caprate/laurate, glyceryl tricaprylate/caprate, triacetin, propylene glycol di-(2-ethylhexanoate), glyceryl tricaprylate/caprate/linoleate, glyceryl tricaprate, glyceryl tricaprylate, and glyceryl triundecanoate.

The liquid oil phase also can comprise a naturally-derived liquid oil, such as corn oil, coconut oil, sunflower seed oil, vegetable oil, cottonseed oil, mineral oil, peanut oil, sesame oil, soybean oil, and/or olive oil. Other oils can be used with the present invention including, but not limited to, liquid fatty alcohols, liquid fatty acids, liquid fatty esters, and phospholipids.

Various MIGLYOL oils have been previously utilized in emulsions or nanoparticle compositions (Sadurni et al., 2005; Fresta et al., 1996; Alonso et al., 2000; EP0711556A1; EP0711557A1 (also published as U.S. Pat. No. 5,658,898); El-Laithy, 2008; Sadurni et al., 2005; DE19852245; EP0865792; Montasser et al., 2003; Alonso et al., 2000; Alonso et al., 1999; WO9904766; Hubert et al., 1989; Al Khouri et al., 1986). However, these compositions lack either the use of both a surfactant and a co-surfactant, and/or one or more physical property of nanoemulsions or nanoparticles of the present invention (e.g., ability to be lyophilized and subsequently re-hydrated while retaining an average particle size of less than about 300 nm).

### B. Surfactants

As used herein, a “surfactant” refers to a surface-active agent, including substances commonly referred to as wetting agents, detergents, dispersing agents, or emulsifying agents. For the purposes of this invention, it is preferred that the surfactant has an HLB value of about 6-20, including an HLB value of about 6, 7, 8, 9, 10, 11, 12, 13, 14, 15, 16, 17, 18, 19, 20, and fractions thereof, and most preferred that the surfactant has an HLB value of about 8-18, including an HLB value of about 8, 9, 10, 11, 12, 13, 14, 15, 16, 17, 18, and fractions thereof. The surfactant and/or co-surfactant can be non-ionic, ionic, or cationic and is selected from the group consisting of polyoxyethylene alkyl ethers, polyoxyethylene sorbitan fatty acid esters, phospholipids, polyoxyethylene stearates, fatty alcohols and their derivatives, hexadecyltrimethylammonium bromide, and combinations thereof.

A surfactant used with the present invention can be chemically modified with a molecule (e.g., polyethylene glycol and polyoxyethylene) to promote increased circulation durations in the blood. Additionally, it is envisioned that the surfactants can be chemically modified with a cell-targeting ligand, such as a small molecule, peptide, protein, or carbohydrate. Surfactants of the present invention are preferably pharmaceutically acceptable surfactants that result in little or no toxicity when administered to a subject according to the present invention. Surfactants are well known in the art and can be found in Remington: The Science and Practice of Pharmacy (21^{st} Edition) Lippincott Williams & Wilkins, or Handbook of Pharmaceutical Excipients (6^{th} Edition) Edited by Raymond C. Rowe, Paul J. Sheskey, and Marian E. Quinn.

A “co-surfactant” refers to a surface-active agent, including substances commonly referred to as wetting agents, detergents, dispersing agents, or emulsifying agents. It is preferred, but not required, that the co-surfactant is selected from the group consisting of: polyoxyethylene alkyl ethers, polyoxyethylene sorbitan fatty acid esters, polyoxyethylene stearates, fatty alcohols or their derivatives, and hexadecyltrimethyl-ammonium bromide, and combinations thereof.

The total concentration of surfactant and/or co-surfactant present in both the oil-in-water microemulsion precursor and the cured nanoparticles system is in the range of about 0.1-50 mM, 0.5-15 mM, or 1-8 mM. For example, the surfactant concentration used in certain nanoparticles in the Examples herein below is about 4 mM (e.g., BRIJ 78=3 mM and TPGS=1 mM).

In certain embodiments the surfactant and/or the co-surfactant are selected from d-alpha-tocopheryl polyethylene glycol 1000 succinate (TPGS) or polyoxyethylene 20-stearyl ether (BRIJ 78). BRIJ 78 has been previously used in various emulsion compositions (Liu et al., 2008).

### C. Cryoprotectants

A cryoprotectant can be included or excluded from a nanoemulsion particle or nanocapsule composition of the present invention, as desired. Cryoprotectants are well known in the art and can be used to protect nanoparticles from the stresses of freezing and thawing (see, e.g., Jeong et al., 2006). Cryoprotectants that can be used with the present invention include sucrose, maltose, mannitol, lactose, trehalose, dextrans, and polyvinyl pyrollidone. In certain embodiments, the inclusion of a cryoprotectant is not required in nanocapsules or nanoemulsion particles of the present invention, which can display increased stability without the presence of a cryoprotectant, e.g., during freezing or lyophilization.

### D. Nanoemulsion Particles and Nanocapsules Comprising Bioactive Agents

The nanoemulsion particles or nanocapsules of the present invention can comprise a bioactive agent. As used herein, the term “bioactive agent” includes, but is not limited to, any agent that has a desired effect on a living cell, tissue, or organism, or an agent that can desirably interact with a component (e.g., enzyme) of a living cell, tissue, or organism, including, but not limited to, polynucleotides, polypeptides, polysaccharides, organic and inorganic small molecules. The term “bioactive agent” encompasses both naturally occurring and synthetic bioactive agents. The term “bioactive agent” also can refer to a detection or diagnostic agent that interacts with a biological molecule to provide a detectable readout that reflects a particular physiological or pathological event. More particularly, in some embodiments, the bioactive agent can include a small molecule, a therapeutic agent, an anti-viral agent, a bacteriostatic or anti-bacterial agent, an anti-fungal agent, a cell-targeting ligand, a peptide, a protein, a carbohydrate, a diagnostic agent, and a viral or bacterial protein capable of eliciting a humoral or cellular-based immune response. For example, when the bioactive agent comprises viral protein capable of eliciting a humoral or cellular-based immune response, the presently disclosed nanocapsule or nanoemulsion particles can comprise a vaccine.

In some embodiments, one or more bioactive agents can be substantially comprised in the liquid oil core of the nanocapsule or the nanoemulsion particle. In yet another embodiment, one or more bioactive agents can be conjugated to the surface of the presently disclosed nanocapsules or nanoemulsion particles. In some embodiments, the one or more bioactive agents can be conjugated directly to the surface of the nanocapsule or nanoemulsion particle, e.g., conjugated to the surfactant or co-surfactant. In other embodiments, the bioactive agent can be conjugated to the nanocapsule or nanoemulsion particle through a linker, for example, through a polyethylene glycol (PEG) or polyoxyethylene moiety. Further, it is contemplated that the presently disclosed nanocapsules and nanoemulsion particles can comprise more than one bioactive agent. For example, in some embodiments, a first bioactive agent, e.g., a therapeutic agent, can be substantially comprised in the liquid oil core of the nanocapsule or nanoemulsion particle, whereas a second bioactive agent, e.g., a cell-targeting ligand, can be conjugated with the surface of the nanocapsule or nanoemulsion particle. Various combinations of a plurality of bioactive agents comprised in liquid oil core and/or conjugated with the surface of the nanocapsules or nanoemulsion particles are thus encompassed by the presently disclosed subject matter.

More particularly, due to the liquid oil phase present in various nanocapsules or nanoemulsion particles of the present invention, substantially water-insoluble or lipophilic bioactive agents, e.g., a therapeutic agent, can be advantageously included in nanoemulsion particles or nanocapsules of the present invention. In various embodiments, the entrapment efficiency of the therapeutic agent in the nanoemulsion particles or nanocapsules can be is at least 50%, at least 75%, at least 85%, or at least 90% in the nanocapsules or nanoemulsion particles. The therapeutic agent can be present in the nanocapsules or nanoemulsion particles at a weight ratio of at least 6% of the liquid oil phase.

Therapeutic agents that can be used with the nanoparticles of the present invention include chemotherapeutic agents, such as lipophilic chemotherapeutic agents (e.g., paclitaxel, and the like). As shown in the below examples, various nanocapsules or nanoemulsion particles of the present invention can be lyophilized and subsequently rehydrated without substantially affecting the potency, e.g., in vitro or in vivo cytotoxicity, of the nanocapsules or nanoemulsion particles as compared to the nanocapsules or nanoemulsion particles prior to lyophilization.

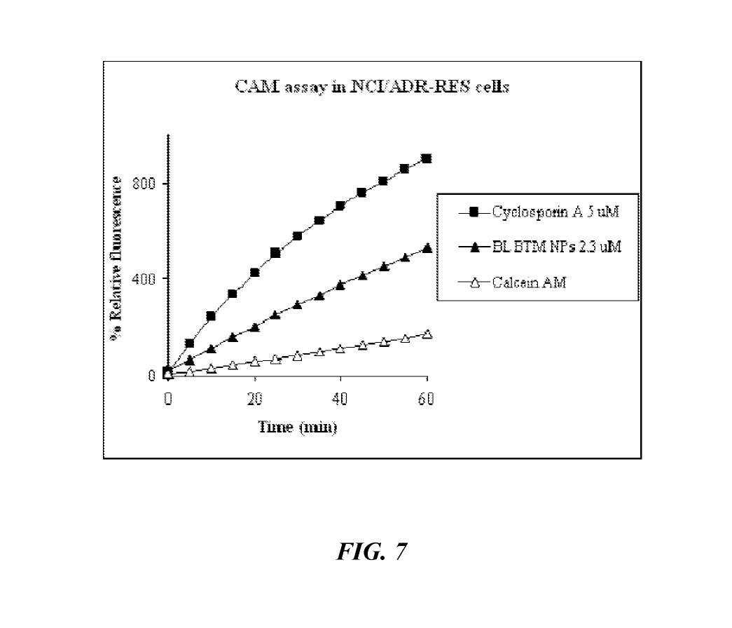

Further, nanoparticles and nanoemulsion particles of the present invention can be used to deliver a chemotherapeutic agent to cells to overcome chemotherapeutic resistance in the cells. As described in more detail herein below in Example 9 and as exemplified in FIGS. 7-9, the presently disclosed nanoemulsion particle or nanocapsule formulations have been found to overcome P-gp mediated resistance in human cancer cells.

1. Lipophilic Therapeutic Agents

A lipophilic therapeutic agent can be included in nanoemulsion or nanocapsule compositions of the present invention. “Lipophilic or “hydrophobic,” as used herein, refers to the physical property of a substance to preferentially associate with or dissolve in organic solvents, such as octanol and/or to repel or not associate with water. Various methods for determining the hydrophobicity or lipophilicity of a substance are known in the art. For example the log₁₀ P of a compound can be measured, wherein P is the partition coefficient (i.e., [concentration dissolved in octanol]/[concentration dissolved in water]). According to this test, when P is less than 0, the compound is considered hydrophilic; when P is greater than 0, the compound is considered hydrophobic.

“Hydrophilic,” as used herein, refers to the physical property of a substance to have a preferential affinity for, dissolve in, or physically associate with water. Hydrophilic interactions can involve hydrogen bonding, dipole-dipole, or a charged interaction with water. The hydrophilicity of a compound can be measured as described immediately hereinabove.

In various embodiments, the nanoemulsion, nanoemulsion particle, and/or nanocapsule compositions of the present invention can comprise or be used to deliver to a subject a lipophilic drug, a lipophilic imaging agent, and/or a lipophilic therapeutic agent.

2. Anti-Cancer Agents

Nanoparticles offer an alternative delivery system for disease therapies, and nanoparticles can be particularly useful in treating cancer. Nanoparticles have the potential to control drug release rates, improve drug pharmacokinetics and biodistribution, and reduce drug toxicities. Due to their small size, nanoparticles comprising entrapped drugs can penetrate tumors due to the discontinuous and leaky nature of the microvasculature of tumors (Pasqualini et al., 2002; Hobbs et al., 1998). Also, the characteristically poor lymphatic drainage of tumors can result in slower clearance of nanoparticles that accumulate in tumors. This well known effect is referred to as the “enhanced permeability and retention” (EPR) effect (Muggia, 1999; Maeda et al., 2001).

In certain embodiments, nanoemulsion particles and/or nanocapsules of the present invention comprise a cancer therapeutic or chemotherapeutic compound. In certain embodiments, substantially lipophilic chemotherapeutic agents can be used with the present invention and administered to a patient, e.g., parenterally. Chemotherapeutic agents that can be used with the present invention include, but are not limited to, nucleic acids (such as RNA and DNA), alkylating agents, anti-metabolites, plant alkaloids and terpenoids, vinca alkaloids, podopyllotoxin, taxanes, topoisomerase inhibitors, antitumor antibiotics, monoclonal antibodies, and hormones.

a. Paclitaxel Nanoparticles

Paclitaxel is an example of a hydrophobic chemotherapeutic agent that can be included in nanoemulsion particles or nanocapsules of the present invention. Paclitaxel is one of the most effective anticancer agents used in the treatment of various tumors. It is a taxane that interferes with microtubule depolymerization in tumor cells resulting in an arrest of the cell cycle in mitosis followed by the induction of apoptosis. However, the high lattice energy of paclitaxel results in very limited aqueous solubility (approximately 0.7-30 μg/mL) (Mathew et al., 1992; Swindell and Krauss, 1991) contributing to only two commercialized dosage forms of injectable paclitaxel, TAXOL and ABRAXANE.

In contrast to certain commercially available forms of paclitaxel, the nanocapsules or nanoemulsion particles of the present invention preferably do not comprise polyethoxylated castor oil. Specifically, TAXOL is composed of a 50:50 (v/v) mixture of CREMOPHOR EL (polyethoxylated castor oil) and dehydrated alcohol, and serious side effects, such as hypersensitivity reactions, attributable to CREMOPHOR EL have been reported (Weiss et al., 1990). Polyethoxylated castor oil can thus be advantageously excluded in nanoemulsion particles or nanocapsules of the present invention.

As shown in the below examples, nanoparticles with liquid oil cores comprising paclitaxel display certain superior characteristics as compared to solid-core nanoparticles comprising paclitaxel. Engineering of stable solid lipid-based nanoparticles from oil-in-water (o/w) microemulsion precursors has been performed. Nanoparticles (E78 NPs) utilizing emulsifying wax (E. wax) as the lipid matrix and BRIJ 78 as the surfactant were reproducibly prepared with particle sizes less than 150 nm. These E78 NPs were found to have excellent hemocompatibility (Koziara et al., 2005) and were shown to be metabolized in vitro by horse liver alcohol dehydrogenase (HLADH)/NAD⁺ (Dong and Mumper, 2006). Paclitaxel (PX) E78 NPs were shown to overcome Pgp-mediated tumor resistance in-vitro in a human HCT-15 colon adenocarcinoma cell line (Koziara et al., 2006) and in vivo in athymic nude mice bearing solid HCT-15 xenograft tumors (Koziara et al., 2006). However, a shortcoming of the PX E78 NPs used in the above examples was that the entrapment efficiency of paclitaxel in the NPs was only 50%, which resulted in relatively rapid in-vitro release (over 80% in 8 hr). These shortcomings were directly attributable to the relatively poor solubility of PX in the melted E. Wax.

As shown in the below examples, the presently disclosed subject matter provides CREMOPHOR-free lipid-based paclitaxel nanoparticle formulations that: 1) use acceptable liquid oil phases having improved solvation ability for PX; 2) display a PX entrapment efficiency greater than 80% with a minimum final concentration of 150 μg/mL with over 5% drug loading; 3) result in slower release profiles of PX from nanoparticles; and 4) display comparable in vitro cytotoxicity as compared to TAXOL.

Two medium-chain triglycerides, glyceryl tridodecanoate and MIGLYOL 812, were selected as the oil phases to engineer nanoparticles from o/w microemulsion precursors. Triglycerides are biocompatible/biodegradable excipients (Traul et al., 2000). It has been reported that paclitaxel has a high partition coefficient (Kp) in medium-chain triglycerides (Dhanikula et al., 2007). Glyceryl tridodecanoate is solid at room temperature, whereas MIGLYOL 812 is liquid at room temperature. Thus, the use glyceryl tridodecanoate and MIGLYOL 812 as oil phases can result in the formation of solid lipid nanoparticles and nanocapsules having a liquid core, respectively. Simplex optimization or the combination of Taguchi array and sequential simplex optimization was used to identify optimized systems based on initial response variables (criteria) of particle size and polydispersity index. Identified leads were then fully characterized for stability, entrapment efficiency, in vitro release, and cytotoxicity in human MDA-MB-231 breast cancer cells.

As shown in the below examples, Sequential Simplex Optimization has been utilized to identify promising new lipid-based paclitaxel nanoparticles having useful attributes. More particularly, to identify and optimize new nanoparticles, experimental design was performed combining Taguchi array and sequential simplex optimization. The combination of Taguchi array and sequential simplex optimization efficiently directed the design of paclitaxel nanoparticles. As shown immediately herein below, CREMOPHOR-free lipid-based paclitaxel (PX) nanoemulsion or nanocapsule formulations were produced from warmed microemulsion precursors.

Two optimized paclitaxel nanoparticles (NPs) were obtained: G78 NPs composed of glyceryl tridodecanoate (GT) and polyoxyethylene 20-stearyl ether (BRIJ 78), and BTM NPs composed of MIGLYOL 812, BRIJ 78 and d-alpha-tocopheryl polyethylene glycol 1000 succinate (TPGS). Both nanoparticles successfully entrapped paclitaxel at a final concentration of 150 μg/mL (over 6% drug loading) with particle sizes less than 200 nm and over 85% of entrapment efficiency. These novel paclitaxel nanoparticles were stable at 4° C. over five months and in PBS at 37° C. over 102 hours as measured by physical stability. Release of paclitaxel was slow and sustained without initial burst release. Cytotoxicity studies in MDA-MB-231 cancer cells showed that both nanoparticles have similar anticancer activities compared to TAXOL. Interestingly, PX BTM nanocapsules could be lyophilized without cryoprotectants. The lyophilized powder comprised only of PX BTM NPs in water could be rapidly rehydrated with complete retention of original physicochemical properties, in vitro release properties, and cytotoxicity profile.

b. Other Chemotherapeutic Agents

Other chemotherapeutic agents that can be used with the present invention include: alkylating agents, cisplatin (CDDP), carboplatin, oxaliplatin, mechlorethamine, cyclophosphamide, chlorambucil, anti-metabolites, plant alkaloids and terpenoids, taxanes, vinca alkaloids (e.g., vincristine, vinblastine, vinorelbine, and vindesine), podophyllotoxin, etoposide, teniposide, taxanes (e.g., docetaxel), topoisomerase inhibitors (e.g., camptothecins, such as irinotecan or topotecan; amsacrine, etoposide, etoposide phosphate, and teniposide), antitumour antibiotics (e.g., dactinomycin), hormones, steroids (e.g., dexamethasone), finasteride, tamoxifen, gonadotropin-releasing hormone agonists (GnRH), such as goserelin, protein-bound paclitaxel (e.g., ABRAXANE), doxorubicin, daunorubicin, mitomycin, actinomycin D, bleomycin, tumor necrosis factor (TNF; cachectin), TAXOL, carmustine, melphalan, cyclophosphamide, chlorambucil, busulfan, and lomustine. 5-fluorouracil, anthocyanin, bleomycin, busulfan, camptothecin, capecitabine, carboplatin, chlorambucil, cyclophosphamide, dactinomycin, estrogen receptor binding agents, etoposide (VP16), farnesyl-protein transferase inhibitors, gemcitabine, idarubicin, ifosfamide, lapatinib, lectrozole, mechlorethamine, melphalan, mitomycin, navelbine, nitrosurea, other platinum containing compounds, parthenolide, plicomycin, a polyphenolic agent derived from nature, procarbazine, raloxifene, tamoxifen, temazolomide (an aqueous form of DTIC), transplatinum, and methotrexate, or any analog or derivative variant of the foregoing. These agents or drugs are categorized by their mode of activity within a cell, for example, whether and at what stage they affect the cell cycle. Alternatively, an agent can be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

3. Other Therapeutic Agents

It is envisioned that a wide variety of therapeutic agents can be included in nanoparticles or nanoemulsion particles of the present invention. It will generally be recognized that therapeutic agents that are substantially water-insoluble or lipophilic can be advantageously administered in compounds of the present invention.

Examples of therapeutic agents that can be used with the present invention include, but are not limited to, agents for the prevention of restenosis, agents for treating renal disease, agents used for intermittent claudication, agents used in the treatment of hypotension and shock, angiotensin converting enzyme inhibitors, antianginal agents, anti-arrhythmics, anti-hypertensive agents, antiotensin ii receptor antagonists, antiplatelet drugs, β-blockers β1 selective, beta blocking agents, botanical products for cardiovascular indications, calcium channel blockers, cardiovascular/diagnostics, central alpha-2 agonists, coronary vasodilators, diuretics and renal tubule inhibitors, neutral endopeptidase/angiotensin converting enzyme inhibitors, peripheral vasodilators, potassium channel openers, anticonvulsants, antiemetics, antinauseants, anti-parkinson agents, antispasticity agents, cerebral stimulants, drugs to treat head trauma, drugs to assist with memory (e.g., to treat Alzheimer's/senility/dementia), drugs to treat migraine, drugs to treat movement disorders.

Also included for use with the present invention are drugs to treat a disease, such as multiple sclerosis, narcolepsy/sleep apnea, stroke, tardive dyskinesia; chronic graft versus host disease, eating disorders, learning disabilities, minimal brain dysfunction, obsessive compulsive disorder, panic, alcoholism, drug abuse, developmental disorders, diabetes, benign prostate disease, sexual dysfunction, rejection of transplanted organs, xerostomia, AIDS patients with Kaposi's syndrome; antineoplastic hormones, biological response modifiers for cancer treatment; also included are vascular agents, cytoxic alkylating agents, cytoxic antimetabolics, cytoxics, immunomodulators, multi-drug resistance modulators, radiosensitizers, anorexigenic agents/CNS stimulants, antianxiety agents/anxiolytics, antidepressants, antipsychotics/schizophrenia, antimanics, sedatives and hypnotics, enkephalin analgesics, hallucinogenic agents, narcotic antagonists/agonists/analgesics, analgesics, epidural and intrathecal anesthetic agents, general, local, regional neuromuscular blocking agents sedatives, preanesthetic adrenal/acth, anabolic steroids, dopamine agonists, growth hormone and analogs, hyperglycemic agents, hypoglycemic agents, large volume parenterals (lvps), lipid-altering agents, nutrients/amino acids, nutritional lvps, obesity drugs (anorectics), somatostatin, thyroid agents, vasopressin, vitamins other than d, antiallergy nasal sprays, antiasthmatic dry powder inhalers, antiasthmatic metered dose inhalers, antiasthmatics (nonsteroidal), (antihistamines, antitussives, decongestants, and the like), beta-2 agonists, bronchoconstrictors, bronchodilators, cough-cold-allergy preparations, inhaled corticosteroids, mucolytic agents, pulmonary anti-inflammatory agents, pulmonary surfactants, anticholinergics, antidiarrheals, antiemetics, cathartics and laxatives, cholelitholytic agents, gastrointestinal motility modifying agents, h2 receptor antagonists, inflammatory bowel disease agents, irritable bowel syndrome agents, liver agents, metal chelators, miscellaneous gastric secretory agents, miscellaneous gi drugs (including hemorrhoidal preparations), pancreatitis agents, pancreatic enzymes, prostaglandins, prostaglandins, gi, proton pump inhibitors, sclerosing agents, sucralfate, anti-progestins, contraceptives, oral contraceptives, estrogens, gonadotropins, gnrh agonists, gnrh antagonists, oxytocics, progestins, uterine-acting agents, anti-anemia drugs, anticoagulants, antifibrinolytics, antiplatelet agents, antithrombin drugs, coagulants, fibrinolytics, hematology, heparin inhibitors (including protamine sulfate and heparinase), blood drugs (e.g., drugs for hemoglobinopathies, hrombocytopenia, and peripheral vascular disease), prostaglandins, vitamin k, anti-androgens, androgens/testosterone, aminoglycosides, antibacterial agents, sulfonamides, antibiotics, antigonorrheal agents, anti-resistant antimicrobials, antisepsis immunomodulators, antitumor agents, cephalosporins, clindamycins, dermatologics, detergents, erythromycins, macrolides, anti-infectives (topical), other systemic antimicrobial drugs, otic-antibiotic in combination, penem antibiotics, penicillins, peptides—antibiotic, sulfonamides, systemic antibiotics, immunomodulators, immunostimulatory agents, aminoglycosides, anthelmintic agents, antibacterial (bacterial vaginosis), antibacterial-quinolones, antifungal (candidiasis), antifungal, systemic, anti-infectives/systemic, antimalarials, antimycobacterial, antiparasitic agents, antiprotozoal agents, antitrichomonads, antituberculosis, chronic fatigue syndrome, immunomodulators, immunostimulatory agents, macrolides, other drugs, including drugs for AIDS related illnesses, other antiparasitic antimicrobial drugs, spiramycin, systemic antibiotics anti-gout drugs, corticosteroids, systemic, cyclooxygenase inhibitors, enzyme blockers, immunomodulators for rheumatic diseases, metalloproteinase inhibitors, nonsteroidal anti-inflammatory agents, antifungals, antihistamines, contraceptives, detergents, non-narcotic analgesics, NSAIDS, vitamins, analgesics, normarcotic, antipyretics, counterirritants, muscle relaxant, anticaries preparations, antigingivitis agents, antiplaque agents, antifibrinolytics, chelating agents, alpha adrenergic agonists/blockers, antibiotics, antifungals, antiprotozoals, antivirals, beta adrenergic blockers, carbonic anhydrase inhibitors, corticosteroids, immune system regulators, mast cell inhibitors, nonsteroidal anti-inflammatory agents, prostaglandins, and proteolytic enzymes.

Examples of diagnostic agents include, but are not limited to, magnetic resonance image (MRI) enhancement agents, positron emission tomography products, radioactive diagnostic agents, radioactive therapeutic agents, radio-opaque contrast agents, radiopharmaceuticals, ultrasound imaging agents, and angiographic diagnostic agents.

In a representative, non-limiting example, as disclosed herein below in Example 14, the presently disclosed BTM nanoparticles were labeled with a gadolinium-diethylenetriaminepentaacetic acid complex to form BTM-DTPA-Gd nanoparticles for use as a contrast agent for MRI imaging.

### E. Design of Nanoparticle Compositions Using Sequential Simplex Optimization and Taguchi Optimization

The combination of Taguchi array and sequential simplex optimization can be used to optimize nanoparticles of the present invention. It will readily be recognized by one of skill in the art that it can be possible to alter one or more of the liquid oil phase, the surfactant, or the co-surfactant to produce nanocapsules or nanoemulsions with substantially the same advantages.

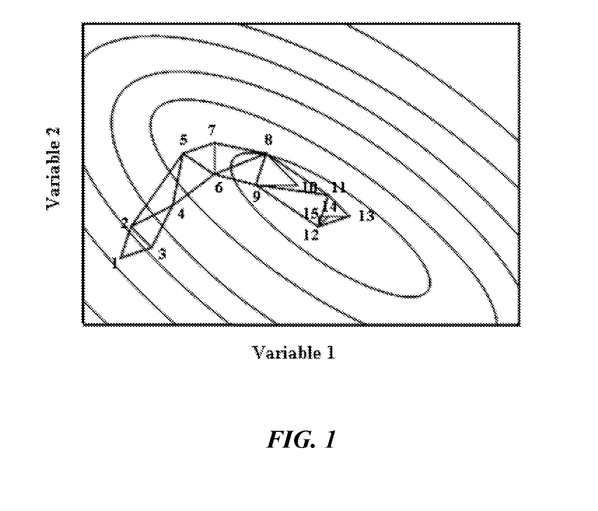

Experimental design is a statistical technique used to simultaneously analyze the influence of multiple factors on the properties of the system being studied. The purpose of experimental design is to plan and conduct experiments to extract the maximum amount of information from the collected data in the smallest number of experimental runs. Factorial design based on a response surface method has been applied to design formulations (Gohel and Amin, 1998; Bhaysar et al., 2006). However, an increase in the number of factors markedly increases the number of experiments to be carried out. The so-called Taguchi approach proposes a special set of orthogonal arrays to standardize fractional factorial designs (Roy, 2001). By this approach, the size of factorial design was reduced. As shown in FIG. 1, sequential simplex optimization is a step-wise strategy for optimization that can adjust many factors simultaneously to rapidly achieve optimal response. The optimization is preceded by moving of a geometric figure (the “simplex”). The starting simplex is composed of k+1 vertex (experiments) wherein k is the number of variables. Then, the experiments are performed one by one. The new simplex is obtained based on the results from the previous simplex and the procedure is repeated until the simplex has rotated and an optimum is encircled. The variable-size simplex algorithm is the modified simplex algorithm that allows the simplex to change its size during movement (FIG. 1). For detailed principles and applications, see Gabrielsson et al., 2002; Walters et al., 1991). Thus, this process of sequential simplex optimization allows for simultaneous formulation development and optimization.

### II. Methods of Making Nanoemulsions, Nanoemulsion Particles and Nanocapsules

The present invention also provides methods for making nanoemulsions, nanoemulsion particles, and nanocapsules. As would be appreciated by one of skill in the art in the art, the preparation of nanoparticles typically involves the use of high-pressure homogenization, microfluidization, high torque mixing, high-pressure mechanical agitation and/or heating. In contrast to these methods, the inventors have discovered that the nanocapsules or nanoemulsion particles of the present invention can be produced without additional heating. This discovery is particularly important as it relates to the possible inclusion of thermosensitive compounds, such as proteins, nucleic acids, and the like, in the nanoemulsion particles or nanocapsules. In embodiments where heating would not be detrimental to the composition, nanoparticles of the present invention can be produced with heating without any additional high pressure mechanical agitation or high torque mixing.

Nanoparticles can be produced using an oil phase, a surfactant, a co-surfactant, and an aqueous solvent or a non-aqueous solvent by heating and subsequently cooling the microemulsion precursor composition. The aqueous solvent can include, for example, water, an aqueous solution comprising 10% lactose, a 150 mM NaCl aqueous solution, and the like.

In certain embodiments, the following protocol can be used to produce nanoparticles of the present invention. Nanoparticles can be prepared from warm oil in water (o/w) microemulsion precursors as previously described with some modification (Oyewumi and Mumper, 2002). Defined amounts of oil phases and surfactants can be weighed into glass vials and heated to 65° C. A desired amount of filtered and deionized (D.I.) water pre-heated at 65° C. (e.g., about 1 mL or similar volumes) can be added into the mixture of melted or liquid oils and surfactants. The mixture can be stirred for 20 min at 65° C. and then cooled to room temperature. To prepare nanoparticles containing a therapeutic agent, the therapeutic agent (e.g., paclitaxel) can be dissolved in a solvent (e.g., ethanol) and added directly to the melted or liquid oil and surfactant. The solvent, e.g., ethanol, can be removed by N₂ stream prior to initiating the process described above.

A nanoemulsion particle or nanocapsule formulation also can be made without heating. In certain embodiments, the following protocol can be used. A liquid oil phase, surfactant, and co-surfactant (e.g., 2.5 mg of MIGLYOL 812, 1.5 mg of TPGS and 3.5 mg of BRIJ 78) can be mixed/dissolved in ethanol. The ethanol was evaporated and water (e.g., about 1 mL) can be added. The system can be mixed overnight at room temperature. In other embodiments, the following protocol can be used. A liquid oil phase 5 mg MIGLYOL 612 and 5 mg Vitamin E TPGS can be mixed/dissolved in ethanol. The ethanol can be evaporated and water (e.g., about 2 mL) can be added. The system can be mixed for 20 minutes at room temperature.

In embodiments where heating is not used to produce nanocapsules or nanoemulsion particles of the present invention, admixing of an oil phase, a surfactant, and a co-surfactant can be performed at ambient temperatures (e.g., less than about 115° F., between about 65-85° F., or between about 70-75° F.).

Some additional time can be required for admixing the components to form nanoparticles or nanocapsules when heating is not used; however, these approaches can be advantageously used, e.g., when a practitioner wishes to include a thermosensitive compound or therapeutic agent in the nanoparticles or nanocapsules. Thermosensitive compounds and therapeutic agents are well known in the art and include various proteins, peptides, nucleic acids, and other molecules whose function can be diminished (e.g., by denaturation, and the like) due to increased temperatures. Additionally, these methods can be advantageously used for thermosensitive compounds that can include small molecules, markers, imaging agents, gene therapies, proteins, enzymes, peptides, and nucleic acids, such as RNA and/or DNA.

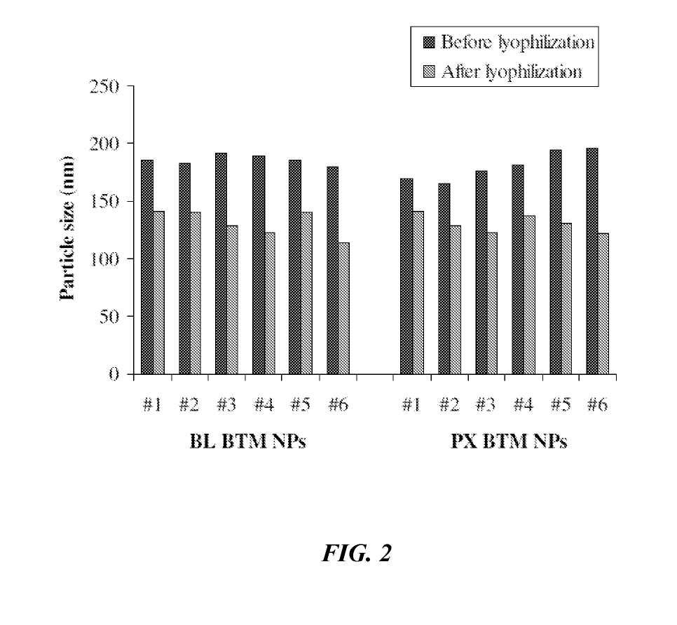

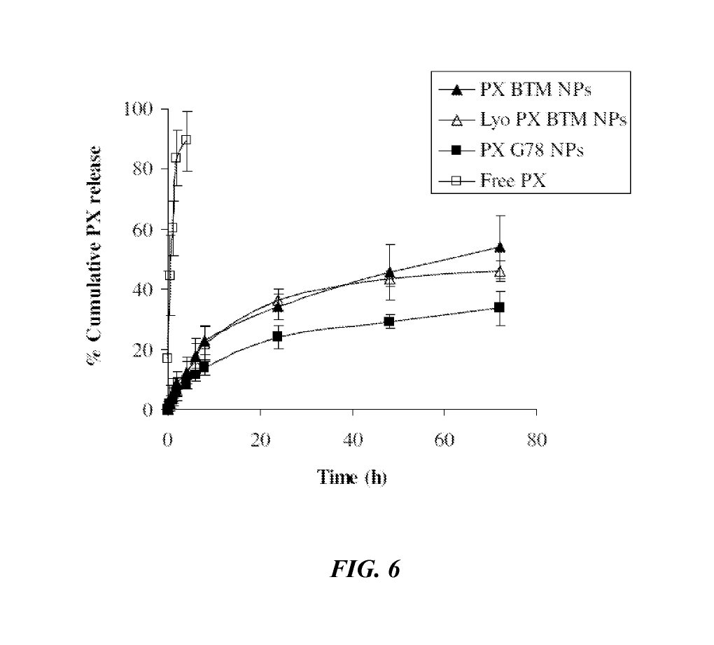

Certain nanoemulsion particles and nanocapsules of the present invention can be lyophilized and subsequently re-hydrated without any increases in particle size and/or without any reduction in the potency or efficacy of a therapeutic agent (e.g., paclitaxel) present in the compositions. As shown in the below examples, lyophilization of various nanoparticles of the present invention in water alone resulted in the formation of dry white cakes that were rapidly rehydrated with water within less than 15 seconds to produce clear nanoparticle suspensions, wherein the nanoparticles showed complete retention of original physicochemical properties and in vitro release properties (FIG. 2 and FIG. 6).

In various embodiments, paclitaxel can be included in nanocapsules or nanoemulsion particles comprising an oil-phase (e.g., a mono-, di-, or triglyceride, a diester propylene glycol), a surfactant and a co-surfactant (TPGS and BRIJ 78). In various embodiments, the following relative amounts of components can be used to produce whatever final quantity of nanoparticles is desired: 450 μg paclitaxel, 7.5 mg of MIGLYOL 812, 4.5 mg of TPGS and 10.5 mg of BRIJ 78 can be mixed at 65° C., and then 1 mL water can be added; 600 μg paclitaxel, 10.0 mg of MIGLYOL 812, 6.0 mg of TPGS and 14.0 mg of BRIJ 78 can be mixed at 65° C., and then 1 mL water can be added; and 750 μg paclitaxel, 12.5 mg of MIGLYOL 812, 7.5 mg of TPGS and 17.5 mg of BRIJ 78 can be mixed at 65° C., and then 1 mL water can be added. After 20 min mixing at 65° C., the system can be cooled to room temperature. The concentration of paclitaxel in the nanocapsule suspension can be evaluated before and after filtration through a 0.2 micron filter. Thus, a 0.2 μm on-line filter possible can be used for intravenous (i.v.) injection.

In certain embodiments, preparation of long-circulating nanoemulsion particles or nanocapsules can be accomplished via the following protocol, and using the following relative amounts (i.e., the quantities can be adjusted to yield whatever final amounts of product are desired). A two (2) mL suspension can be prepared from warm o/w microemulsion precursors by adding 2.5 mg of MIGLYOL 812, 1.5 mg of TPGS and 3 mg of BRIJ 78 to a glass vial and heating to 65° C. 975 microliters of filtered and deionized (D.I.) water pre-heated at 65° C. can be added into the mixture of melted oils and surfactants. After 15 min of mixing, 25 microliters of a 8 mg BRIJ 700/mL stock solution can be added to the warm mixture and mixed for an additional 10 min. The mixture can then be cooled to room temperature and stirred for another 5 hr. BRIJ 700, also known as Steareth-100, has a polyethylene glycol (PEG) moiety (Mw of PEG about 4400) and can be added to the formulation to form sterically stabilized nanoparticles to increase circulation times in the blood.

### III. Pharmaceutical Preparations

The nanocapsules or nanoemulsion particles of the present invention can be formulated for administration to a subject, e.g., a human patient, via various routes. For example, the nanocapsules or nanoemulsion particles can be formulated for parenteral, intravenous (i.v.), topical, rectal, oral, inhalation, intranasal, transdermal, or buccal administration. In certain embodiments, a substantially water insoluble or lipophilic drug can be effectively stored and administered parenterally as a nanosuspension. In other embodiments, a nanocapsule or nanoemulsion formulation can be lyophilized or produced in a spray-dried powder. The spray dried powder can subsequently be formulated in an oral dosage forms, such as a compressed tablet or a capsule-based formulation. Thus, in certain embodiments, the compositions of the present invention can be formulated for delivery via an alimentary route. In other embodiments, nanocapsules or nanoemulsion particles of the present invention can be delivered via inhalation (e.g., in an aerosol formulation and the like).

Pharmaceutical compositions of the present invention comprise an effective amount of one or more nanoemulsion particles or nanocapsules of the present invention and can include, in some embodiments, one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one nanoemulsion particle or nanocapsule or additional active ingredient will be known to those of skill in the art in light of the present disclosure, as exemplified by Remington: The Science and Practice of Pharmacy, 21^{st} edition, by University of the Sciences in Philadelphia, incorporated herein by reference. Moreover, for animal (e.g., human) administration, it will be understood that preparations should meet sterility, pyrogenicity, general safety and purity standards as required by, for example, by the FDA's General Biological Products Standards as provided in 21 C.F.R. part 610.

The nanoemulsion particle or nanocapsule compositions can comprise different types of carriers depending on whether it is to be administered in solid, liquid or aerosol form, and whether it is required to be sterile for such routes of administration as injection. The present invention can be administered intravenously, intradermally, intracranially, transdermally, intrathecally, intraarterially, intraperitoneally, intranasally, intravaginally, intrarectally, topically, intramuscularly, subcutaneously, mucosally, orally, locally, inhalation (e.g., aerosol inhalation), injection, infusion, continuous infusion, localized perfusion bathing target cells directly, via a catheter, via a lavage, in cremes, in lipid compositions, or by other method or any combination of the forgoing as would be known to one of ordinary skill in the art (see, for example, Remington's Pharmaceutical Sciences, 18th Ed. Mack Printing Company, 1990, incorporated herein by reference). In certain embodiments, a nanoemulsion particle or nanocapsule composition of the present invention is administered intravenously or parenterally.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in an administrable composition for use in practicing the methods of the present invention is appropriate. Examples of carriers or diluents include fats, oils, water, saline solutions, lipids, liposomes, resins, binders, fillers and the like, or combinations thereof. The composition also can comprise various antioxidants to retard oxidation of one or more component. Additionally, the prevention of the action of microorganisms can be brought about by preservatives, such as various antibacterial and antifungal agents, including, but not limited to, parabens (e.g., methylparabens, propylparabens), chlorobutanol, phenol, sorbic acid, thimerosal or combinations thereof.

### A. Parenteral Compositions and Formulations

In further embodiments, nanoemulsion particle or nanocapsule compositions can be administered via a parenteral route. As used herein, the term “parenteral” includes routes of administration that bypass the alimentary tract. Specifically, the pharmaceutical compositions disclosed herein can be administered for example, but not limited to intravenously, intradermally, intramuscularly, intraarterially, intrathecally, subcutaneous, or intraperitoneally see U.S. Pat. Nos. 6,537,514; 6,613,308; 5,466,468; 5,543,158; 5,641,515; and 5,399,363 (each of which is incorporated herein by reference in its entirety).

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms. The pharmaceutical formulations suitable for injectable use include sterile aqueous solutions or dispersions and sterile powders for the extemporaneous preparation of sterile injectable solutions or dispersions (U.S. Pat. No. 5,466,468, which is incorporated herein by reference in its entirety). In all cases the formulation must be sterile and also must be fluid to the extent to facilitate easy injectability. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage can be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA's General Biological Products Standards as provided in 21 C.F.R. part 610.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques that yield a powder of the active ingredient plus any additional desired ingredient from a previously sterile-filtered solution thereof. A powdered composition is combined with a liquid carrier, such as, e.g., water or a saline solution, with or without a stabilizing agent.

### B. Miscellaneous Pharmaceutical Compositions and Formulations

In other preferred embodiments of the invention, the nanoemulsion particle or nanocapsule composition can be formulated for administration via various miscellaneous routes, for example, oral, topical (i.e., transdermal) administration, mucosal administration (intranasal, vaginal, and the like) and/or inhalation.

Pharmaceutical compositions for topical administration can include the nanoemulsion particle or nanocapsule composition formulated for a medicated application, such as an ointment, gel, paste, cream or powder. Ointments include all oleaginous, adsorption, emulsion and water-soluble based compositions for topical application, while creams and lotions are those compositions that include an emulsion base only. Topically administered medications can contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum, as well as any other suitable absorption, emulsion or water-soluble ointment base. Topical preparations also can include emulsifiers, gelling agents, and antimicrobial preservatives as necessary to preserve the active ingredient and provide for a homogenous mixture. Transdermal administration of the present invention also can comprise the use of a “patch.” For example, the patch can supply one or more active substances at a predetermined rate and in a continuous manner over a fixed period of time.

In certain embodiments, the pharmaceutical compositions can be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each of which is incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) also are well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term “aerosol” refers to a colloidal system of finely divided solid or liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Delivery of Active Agents for the Treatment of Diseases

It is anticipated that the nanocapsule and nanoemulsion particle compositions of the present invention can be used to deliver a bioactive agent, e.g., a therapeutic agent to actively or prophylactically treat a variety of diseases. For example, the nanocapsule and nanoemulsion particle compositions can comprise a drug or therapeutic agent the treatment of cancer, cardiovascular disease, depression, inflammation, diseases of the central nervous system, and/or the prevention or therapy of an infectious disease, such as a bacterial, fungal, viral, or protozoan disease, and the like. The nanocapsule and nanoemulsion particle compositions can comprise a bioactive, e.g., a vaccine, to prophylactically prevent or reduce the incidence of recurrence of a disease.

A. Cancer Therapies

The nanoemulsion particle or nanocapsule compositions of the present invention can be administered to a subject, such as a mammal, a rat, a mouse, a non-human animal, or a human patient, to treat a cancer. Although it is envisioned that the compositions of the present invention can be used to treat virtually any cancer, in certain embodiments, a nanoemulsion particle or nanocapsule comprising an anti-cancer compound can be administered to a subject to treat leukemia, cancer of the lymph node or lymph system, bone cancer, cancer of the mouth and esophagus, stomach cancer, colon cancer, breast cancer, ovarian cancer, a gastric cancer, brain cancer, renal cancer, liver cancer, prostate cancer, melanoma, lung cancer, a tumor, and/or a metastasis.

B. Combination Therapies

To increase the effectiveness of a nanocapsule or nanoemulsion particle composition comprising an anti-cancer compound, e.g., a chemotherapeutic agent, it can be desirable to combine these compositions and methods of the invention with an agent effective in the treatment of a hyperproliferative disease, such as, for example, an anti-cancer agent. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

More generally, such an agent would be provided in a combined amount with a nanoemulsion particle or nanocapsule composition effective to kill or inhibit proliferation of a cancer cell. This process can involve contacting the cell(s) with an agent(s) and the nanoemulsion particle or nanocapsule composition at the same time or within a period of time wherein separate administration of the nanoemulsion particle or nanocapsule composition and an agent to a cell, tissue or organism produces a desired therapeutic benefit. This benefit can be achieved by contacting the cell, tissue, or organism with a single composition or pharmacological formulation that includes both a nanoemulsion particle or nanocapsule composition and one or more agents, or by contacting the cell with two or more distinct compositions or formulations, wherein one composition includes a nanoemulsion particle or nanocapsule composition and the other includes one or more agents.

The terms “contacted” and “exposed,” when applied to a cell, tissue or organism, are used herein to describe the process by which a therapeutic construct of a nanoemulsion particle or nanocapsule composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the nanoemulsion particle or nanocapsule composition and/or additional agent(s) are delivered to one or more cells in a combined amount effective to kill the cell(s) or prevent them from dividing.

The nanoemulsion particle or nanocapsule composition can precede, be co-current with and/or follow the other agent(s) by intervals ranging from minutes to weeks. In embodiments where the nanoemulsion particle or nanocapsule composition, and other agent(s) are applied separately to a cell, tissue or organism, one would generally ensure that a significant period of time did not expire between the time of each delivery, such that the nanoemulsion particle or nanocapsule composition and agent(s) would still be able to exert an advantageously combined effect on the cell, tissue or organism. For example, in such instances, it is contemplated that one can contact the cell, tissue or organism with two, three, four or more modalities substantially simultaneously (i.e. within less than about a minute) as the nanoemulsion particle or nanocapsule composition. In other aspects, one or more agents can be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1 week, about 2 weeks, about 3 weeks, about 4 weeks, about 5 weeks, about 6 weeks, about 7 weeks or about 8 weeks or more, and any range derivable therein, prior to and/or after administering the nanoemulsion particle or nanocapsule composition.

Various combination regimens of the nanoemulsion particle or nanocapsule composition and one or more agents can be employed. Non-limiting examples of such combinations are shown below, wherein a composition of the nanoemulsion particle or nanocapsule composition is “A” and an agent is “B”:

```
A/B/A  B/A/B  B/B/A  A/A/B  A/B/B  B/A/A  A/B/B/B  B/A/B/B
    B/B/B/A  B/B/A/B  A/A/B/B  A/B/A/B  A/B/B/A  B/B/A/A
    B/A/B/A  B/A/A/B  A/A/A/B  B/A/A/A  A/B/A/A  A/A/B/A
```

Administration of the composition of the nanoemulsion particle or nanocapsule composition to a cell, tissue or organism can follow general protocols for the administration of chemotherapeutic agents, taking into account the toxicity, if any. It is expected that the treatment cycles would be repeated as necessary. In particular embodiments, it is contemplated that various additional agents can be applied in any combination with the present invention.

1. Chemotherapeutic Agents

The term “chemotherapy” refers to the use of drugs to treat cancer. A “chemotherapeutic agent” is used to connote a compound or composition that is administered in the treatment of cancer. One subtype of chemotherapy known as biochemotherapy involves the combination of a chemotherapy with a biological therapy. The chemotherapeutic agents described above are examples of chemotherapeutic agents that can be used with the present invention.

Chemotherapeutic agents and methods of administration, dosages, and the like, are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and can be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes also are described herein. Of course, all of these dosages and agents described herein are exemplary rather than limiting, and other doses or agents can be used by a skilled artisan for a specific patient or application. Any dosage in-between these points, or range derivable therein also is expected to be of use in the invention.

2. Radiotherapeutic Agents

Radiotherapeutic agents include radiation and waves that induce DNA damage for example, y-irradiation, X-rays, proton beam therapies (U.S. Pat. Nos. 5,760,395 and 4,870,287), UV-irradiation, microwaves, electronic emissions, radioisotopes, and the like. Therapy can be achieved by irradiating the localized tumor site with the above described forms of radiations. It is most likely that all of these agents affect a broad range of damaged DNA, on the precursors of DNA, the replication and repair of DNA, and the assembly and maintenance of chromosomes.

Radiotherapeutic agents and methods of administration, dosages, and the like, are well known to those of skill in the art, and can be combined with the invention in light of the disclosures herein. For example, dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

3. Surgery

Approximately 60% of persons with cancer will undergo surgery of some type, which includes, for example, preventative, diagnostic or staging, curative and palliative surgery. Surgery, and in particular a curative surgery, can be used in conjunction with other therapies, such as the present invention and one or more other agents.

Curative surgery includes resection in which all or part of cancerous tissue is physically removed, excised and/or destroyed. It is further contemplated that surgery can remove, excise or destroy superficial cancers, precancers, or incidental amounts of normal tissue. Treatment by surgery includes for example, tumor resection, laser surgery, cryosurgery, electrosurgery, and miscopically controlled surgery (Mohs' surgery). Tumor resection refers to physical removal of at least part of a tumor. Upon excision of part of all of cancerous cells, tissue, or tumor, a cavity can be formed in the body.

Further treatment of the tumor or area of surgery can be accomplished by perfusion, direct injection or local application of the area with an additional anti-cancer agent. Such treatment can be repeated, for example, about every 1 day, about every 2 days, about every 3 days, about every 4 days, about every 5 days, about every 6 days, or about every 7 days, or about every 1 week, about every 2 weeks, about every 3 weeks, about every 4 weeks, or about every 5 weeks or about every 1 month, about every 2 months, about every 3 months, about every 4 months, about every 5 months, about every 6 months, about every 7 months, about every 8 months, about every 9 months, about every 10 months, about every 11 months, or about every 12 months. These treatments can be of varying dosages as well.

4. Immunotherapeutic Agents

An immunotherapeutic agent generally relies on the use of immune effector cells and molecules to target and destroy cancer cells. The immune effector can be, for example, an antibody specific for some marker on the surface of a tumor cell. The antibody alone can serve as an effector of therapy or it can recruit other cells to actually effect cell killing. The antibody also can be conjugated to a drug or toxin (e.g., a chemotherapeutic agent, a radionuclide, a ricin A chain, a cholera toxin, a pertussis toxin, and the like) and serve merely as a targeting agent. Such antibody conjugates are referred to immunotoxins, and are well known in the art (see U.S. Pat. Nos. 5,686,072; 5,578,706; 4,792,447; 5,045,451; 4,664,911, and 5,767,072, each of which is incorporated herein by reference in their entirety). Alternatively, the effector can be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these can be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

5. Genetic Therapy Agents

A tumor cell resistant to agents, such as chemotherapeutic agent and radiotherapeutic agents, represents a major problem in clinical oncology. One goal of current cancer research is to find ways to improve the efficacy of one or more anti-cancer agents by combining such an agent with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that gene therapy could be used similarly in conjunction with the nanoemulsion particle or nanocapsule composition and/or other agents.

C. Vaccine Therapies

The presently disclosed nanocapsules or nanoemulsion particles also can be used as a vaccine delivery system. For example, as demonstrated herein below in Example 10, the presently disclosed nanocapsules or nanoemulsion particles can comprise a viral protein capable of eliciting a humoral or cellular-based immune response.

### V. Examples

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Materials and Methods

### Materials and Cell Culture

Paclitaxel, glyceryl tridodecanoate, PBS, and Tween 80 were purchased from Sigma-Aldrich (St. Louis, Mo., United States of America). Emulsifying wax and stearyl alcohol were purchased from Spectrum Chemicals (Gardena, Calif., United States of America). Polyoxyethylene 20-stearyl ether (BRIJ 78) was obtained from Uniqema (Wilmington, Del., United States of America). D-alpha-tocopheryl polyethylene glycol 1000 succinate (TPGS) was purchased from Eastman Chemicals (Kingsport, Tenn., United States of America). MIGLYOL 812 is a mixed caprylic (C_{8:0}) and capric (C_{10:0}) fatty acid triglyceride and was obtained from Sasol Germany GmbH (Witten, Germany). Dialyzers with a molecular weight cutoff (MWCO) of 8000 were obtained from Sigma-Aldrich (St. Louis, Mo., United States of America). Microcon Y-100 with MWCO 100 kDa was purchased from Millipore (Bedford, Mass., United States of America). Ethanol USP grade was purchased from Pharmco-AAPER (Brookfield, Conn., United States of America). TAXOL was obtained from Mayne Pharma Inc. (Paramus, N.J., United States of America). The human breast cancer cell line, MDA-MB-231, was obtained from American Type Culture Collection (ATCC) and was maintained in Dulbecco's Modified Eagle's Medium (DMEM) supplemented with 10% fetal bovine serum (FBS). Cells were cultured at 37° C. in a humidified incubator with 5% CO₂ and maintained in exponential growth phase by periodic subcultivation.

Preparation of Nanoparticles from Microemulsion Precursors

Nanoparticles were prepared from warm o/w microemulsion precursors as previously described with some modification (Oyewumi and Mumper, 2002). Defined amounts of oil phases and surfactants were weighed into glass vials and heated to 65° C. One (1) mL of filtered and deionized (D.I.) water pre-heated at 65° C. was added into the mixture of melted or liquid oils and surfactants. The mixture were stirred for 20 min at 65° C. and then cooled to room temperature. To prepare PX NPs, 150 μg of PX dissolved in ethanol was added directly to the melted or liquid oil and surfactant and ethanol was removed by N₂ stream prior to initiating the process described above. Particle size and size distribution of NPs were measured using a N5 Submicron Particle Size Analyzer (Beckman Coulter, Fullerton, Calif., United States of America). Ten microliters of nanoparticles were diluted with 1 mL of D.I. water to reach within the density range required by the instrument, and particle size analysis was performed at 90° light scattering at 25° C.

### Development of Prototype Nanoparticles by Sequential Simplex Optimization

BTM Nanoparticles Comprised of MIGLYOL 812, BRIJ 78 and TPGS

MIGLYOL 812 and stearyl alcohol were chosen as oil phases, and BRIJ 78 and TPGS were selected as the surfactants. Taguchi array L-9 (3⁴) was first used to help set up the starting simplex for sequential simplex optimization. Three levels for each excipient and Taguchi array are presented in Table 2A. As directed by the results from Taguchi array, trial 3, 5, and 9 were used for the starting simplex (Table 2B). Sequential simplex optimization then was performed as previously described following the variable-size simplex rules (Walters et al., 1991). Desirability functions previously developed for the simultaneous optimization of different response variables (criteria) (Derringer, 1980) were used to evaluate the results using particle size and polydispersity index (P.I.) as the response variables. The resultant particle size and P.I. were transformed to d_{size} and d_{P.I.} within 0-1 interval, respectively

$${d}_{i} = \{ \begin{matrix}0 & {Y}_{i} \leq a \\ {[ \frac{{Y}_{i} - a}{b - a} ]}^{S} & a < {Y}_{i} < b \\ 1 & {Y}_{i} \geq b\end{matrix} \qquad Equation ( 1 )$$

In Equation (1), the variable “i” indicates particle size or P.I. The limits were from a=70 nm to b=250 nm for particle size, and from a=0.05 to b=1.2 for P.I. For these optimization experiments, particle size and P.I. were given equal importance; thus, the constant s=1.

The overall contribution of all responses is presented as a single D value as calculated by Equation (2):

D=(d_{particle size}×d_{P.I.})^{1/2} Equation (2)

After the sequential simplex optimization, MIGLYOL 812, BRIJ 78 and TPGS were chosen to form BTM NPs. Four different compositions based on the results from sequential simplex optimization were tested (Table 2C) wherein two milliliter NP formulations were prepared for each composition.

### G78 Nanoparticles Comprised of Glyceryl Tridodecanoate and Brij 78

G78 nanoparticles were optimized using MultiSimplex software (CambridgeSoft Corporation, Cambridge, Mass., United States of America). The variable-size simplex rules also were used in this optimization, and response variables included particle size, P.I. and the peak numbers in nanoparticle distribution. The limits were from a=50 nm to b=200 nm for particle size, and from a=0.01 to b=0.4 for P.I., and from a=1 to b=2 for peak numbers. Two milliliter NP formulations were prepared for each composition.

### Lyophilization of PX NPs

To determine the effect of lyophilization on the NPs, blank and PX NPs in the presence or absence of 5% lactose were lyophilized using a VIRTIS lyophilizer (SP Industries, Gardiner, N.Y., United States of America). Two milliliters of each sample were rapidly frozen at −40° C. and then lyophilized using a program of 7.5 h at −10° C. for primary drying and 7.5 h at 25° C. for secondary drying at 100 mTorr. The resultant lyophilized products were reconstituted in 2 mL of D.I. water using a plate shaker for 5 min. The particle sizes of reconstituted lyophilized NPs from six different batches were measured as described above.

### Characterization of Paclitaxel G78 and BTM Nanoparticles

### Particle Size and Zeta Potential Measurement

Nanoparticles were analyzed for particle size and size distribution as described above. Ten microliters of blank NPs and PX NPs were diluted with 1 mL of D.I. water and 10 μL of PBS buffer (pH 7.4) was added for measurement of Zeta potentials using Zetasizer Nano ZEN2600 (Malvern Instruments, Worcs, United Kingdom).

### Determination of Drug Loading and Entrapment Efficiency

The concentration of PX was quantified by HPLC using a Thermo Finnigan Surveyer HPLC System and an Inertsil ODS-3 column (4.6×150 mm) (GL Sciences Inc.) preceded by an Agilent guard column (Zorbax SB-C 18, 4.6×12.5 mm). The mobile phase was water-acetonitrile (40:60, v/v) at a flow rate of 1.0 mL/min with PX detection at 227 nm. For the paclitaxel standard curve, paclitaxel was dissolved in methanol. To quantify PX in NPs, 1 part of PX NPs in water were dissolved in 8 parts of methanol. PX BTM NPs containing 30% of 7-epi PX was dissolved in methanol and then serially diluted in methanol to prepare the standard curve of 7-epi PX. Drug loading and entrapment efficiencies were determined by separating free PX from PX-loaded NPs using a Microcon Y-100, and then measuring PX in NP-containing supernatants as described above. To ensure mass balance, the filtrates also were assayed for PX. PX loading and PX entrapment efficiency were calculated as follows:

% drug loading=[(drug entrapped in NPs)/(weight of oil)]×100% (w/w)

% drug entrapment efficiency=[(drug entrapped in NPs)/(total drug added into NP preparation)]×100% (w/w)

### Particle Size Stability of NPs in 4° C. and 37° C.

The physical stability of G78 and BTM nanoparticle suspensions was assessed over storage at 4° C. for five months. Prior to particle size measurement, NP suspensions were allowed to equilibrate to room temperature. The stability of all NP suspensions also was assessed at 37° C. in 10 mM PBS, pH 7.4 by adding 100 μL NP suspensions to 13 mL PBS buffer with a water-bath shaker mixing at 150 rpm. At each time interval, 1 mL aliquots were removed and allowed to equilibrate to room temperature prior to particle size measurement.

### DSC Analysis for G78 NPs

Differential scanning calorimetry (DSC) analysis was performed to determine the physical state of the core (glyceryl tridodecanoate) lipid. Blank G78 or PX G78 nanoparticle suspensions were concentrated about 20-fold using Microcon Y-100 at 4° C. The concentrated NPs were: (1) analyzed by DSC immediately; or (2) transferred to an aluminum pan, which was placed in a desiccator for two days at room temperature prior to DSC analysis. As controls, bulk glyceryl tridodecanoate (5 mg), BRIJ 78 (5 mg) and the bulk mixture of glyceryl tridodecanoate (3.4 mg) and BRIJ 78 (8 mg) were placed in aluminum pans for DSC analysis (PerkinElmer, Norwalk, Conn.). Heating curves were recorded using a scan rate of 1° C./min from 15° C. to 66° C.

### In-Vitro Release Studies

PX release studies (n=4) were completed at 37° C. by the dialysis method using PBS with 0.1% Tween 80 as release medium. Before release studies, the solubility of PX in release medium was measured. Briefly, extra amounts of paclitaxel were added into 2 mL of release medium until saturation was attained. After centrifuge, the concentration of PX in the supernatant was determined by HPLC as described above. For release studies, one milliliter (1 mL) of PX G78 NPs was purified with a Microcon Y-100 and re-suspended into 1 mL D.I. water. The concentration of PX in re-suspended PX G78 NPs was measured by HPLC as described above. Eight hundred microliters of purified PX G78 NPs, PX BTM NPs and reconstituted lyo BTM NPs were placed into a regenerated cellulose dialysis membrane (MWCO 8000 Da) submerged in 40 mL PBS with 0.1% Tween 80, respectively, and then shaken in a water bath at a speed of 150 rpm at 37° C. Free PX also was used as a control. At predetermined times, 200 μL aliquots were taken from outside of the dialysis membrane, and replaced with 200 μL fresh media. PX was measured by HPLC as described above. Mass balance was confirmed by measuring PX concentration inside the dialysis membranes after 72 h. In addition, the particle sizes of PX NPs inside the dialysis membranes were measured when release studies were terminated (at 72 h).

### In-Vitro Cytotoxicity Studies

The cytotoxicity of PX NPs was tested in human MDA-MB-231 breast cancer cells using the sulforhodamine B (SRB) assay (Papazisis et al., 1997). Cells were seeded into 96-well plates at 1.5×10⁴ cells/well and cells were allowed to attach overnight. Cells were incubated for 48 h with drug equivalent concentrations ranging from 10,000 nM to 0.01 nM for TAXOL, PX-loaded NPs and blank NPs. The SRB assay was performed and IC50 values were determined. Briefly, the cell lines were fixed with cold 10% trichloroacetic acid and stained using 0.4% SRB dissolved in 1% acetic acid. The bound dye was solubilized with 10 mM tris base and the absorbance was measured at 490 nm using a microplate reader. IC50 values were calculated based on the percentage of treatment over control. All groups included three independent experiments (N=3) with triplicates (n=3) for each experiment.

### Statistical Analysis

Statistical comparisons were made with ANOVA followed by pair-wise comparisons using Student's t test using GraphPad Prism software. Results were considered significant at 95% confidence interval (p<0.05).

### Example 2

### Development of New Lipid-Based Paclitaxel Nanoparticles Using Sequential Simplex Optimization

Sequential Simplex Optimization was utilized to identify promising new lipid-based paclitaxel nanoparticles having useful attributes. The objective of this Example was to develop CREMOPHOR-free lipid-based paclitaxel (PX) nanoparticle formulations prepared from warm microemulsion precursors. To identify and optimize new nanoparticles, experimental design was performed combining Taguchi array and sequential simplex optimization. The combination of Taguchi array and sequential simplex optimization efficiently directed the design of paclitaxel nanoparticles. Two optimized paclitaxel nanoparticles (NPs) were obtained: (1) G78 NPs composed of glyceryl tridodecanoate (GT) and polyoxyethylene 20-stearyl ether (BRIJ 78); and (2) BTM NPs composed of MIGLYOL 812, BRIJ 78 and d-alpha-tocopheryl polyethylene glycol 1000 succinate (TPGS). Both nanoparticles successfully entrapped paclitaxel at a final concentration of 150 μg/mL (over 6% drug loading) with particle sizes less than 200 nm and over 85% of entrapment efficiency. These novel paclitaxel nanoparticles were stable at 4° C. over three months and in PBS at 37° C. over 102 hours as measured by physical stability. Release of paclitaxel was slow and sustained without initial burst release. Cytotoxicity studies in MDA-MB-231 cancer cells showed that both nanoparticles have similar anticancer activities compared to TAXOL. Interestingly, PX BTM nanocapsules could be lyophilized without cryoprotectants. The lyophilized powder comprised only of PX BTM NPs in water could be rapidly rehydrated with complete retention of original physicochemical properties, in-vitro release properties, and cytotoxicity profile.

### Example 3

### Development of BTM Nanoparticles by Taguchi Array and Sequential Simplex Optimization

It has previously been reported that a combination of liquid and solid lipid oils enhance drug loading and stability in nanoparticles as compared to a only a solid lipid core (Muller and Radtke, 2002; Manjunath et al., 2005). In the initial development of NPs, a combination oil phase of MIGLYOL 812 (liquid oil) and stearyl alcohol (solid oil) were selected, in addition to two potential surfactants, BRIJ 78 and TPGS. Based on these four variables (excipients), Taguchi array was carried out to determine the extent of compositions to which the starting simplex could be formed efficiently.

Taguchi's orthogonal array for 3 levels 4 variables (L-9 3⁴) is shown in Table 2A. As depicted in Table 2A, trials 3, 5 and 9 gave the most promising results. Thus, the compositions of these three trials (3, 5, and 9) were used to construct the starting simplex in the sequential simplex optimization (Table 2B). As described in the methods section, there were two basic criteria for current nanoparticle formulation: particle size (<200 nm) and P.I. (<0.35). The D value from desirability functions including particle size and P.I. as response variables was used to evaluate the result of each experiment. Interestingly, the simplex (trial 6 in Table 2B) identified an initial NP formulation that did not contain stearyl alcohol (the solid oil component), but was comprised of MIGLYOL 812, BRIJ 78 and TPGS. Thus, as directed by sequential simplex optimization, subsequent experiments focused on these three excipients. Four different compositions were used to prepare nanoparticles as shown in Table 2C. Among them, trial 2 resulted in optimized NPs having a mean particle size of 149 nm and P.I. of 0.328. Interestingly, due to the relatively low concentration of the resulting NPs, 150 μg/mL of paclitaxel could not be entrapped into these NPs. However, when each component was increased by a factor of 2.5, the more concentrated NP formulation was able to accommodate the desired concentration of PX. This final BTM NP formulation consisted of 2.5 mg of MIGLYOL 812, 1.5 mg of TPGS and 3.5 mg of BRIJ 78 in 1 mL water with 150 μg/mL of paclitaxel.

TABLE 2A
Taguchi array for the development of BTM nanoparticles.†

| | | | Stearyl | | | |
|---|---|---|---|---|---|---|
| | BRIJ 78 | TPGS | Alcohol | MIGLYOL 812 | Particle Size | |
| Trial | (mg) | (mg) | (mg) | (mg) | (nm) | P.I. |
| 1 | 1.6 | 1.2 | 0.6 | 1.4 | 35 | 1.210 |
| 2 | 1.6 | 0.9 | 0.4 | 1.0 | 193.5 | 0.978 |
| 3 | 1.6 | 0.6 | 0.2 | 0.6 | 118.4 | 0.159 |
| 4 | 1.2 | 1.2 | 0.4 | 0.6 | 25 | 1.435 |
| 5 | 1.2 | 0.9 | 0.2 | 1.4 | 212.9 | 0.307 |
| 6 | 1.2 | 0.6 | 0.6 | 1.0 | 282.6 | 0.897 |
| 7 | 0.7 | 1.2 | 0.2 | 1.0 | 130.5 | 0.826 |
| 8 | 0.7 | 0.9 | 0.6 | 0.6 | 315 | 1.685 |
| 9 | 0.7 | 0.6 | 0.4 | 1.4 | 234.6 | 0.355 |

†Listed are the compositions per 1 mL nanoparticle suspensions.

TABLE 2B
Sequential Simplex Optimization for the
Development of BTM Nanoparticles.†

| | | BRIJ | | Stearyl | MIGLYOL | Particle | | | | |
|---|---|---|---|---|---|---|---|---|---|---|
| | | 78 | TPGS | Alcohol | 812 | Size | | | | |
| Trial | Movement | (mg) | (mg) | (mg) | (mg) | (nm) | P.I. | d_{size} | d_{P.I.} | D |
| 1 | \ | 1.6 | 0.6 | 0.4 | 0.6 | 35.6 | 0.070 | 0 | 0.017 | 0 |
| 2 | \ | 1.2 | 0.9 | 0.4 | 1.4 | 197.6 | 0.449 | 0.709 | 0.347 | 0.496 |
| 3 | \ | 0.7 | 0.6 | 0.8 | 1.4 | 186.3 | 0.360 | 0.646 | 0.270 | 0.417 |
| 4 | \ | 1.6 | 0.9 | 1.6 | 1.2 | 309.2 | 1.079 | 0 | 0.895 | 0 |
| 5 | \ | 0.7 | 2.1 | 1.6 | 1.2 | 182.7 | 1.028 | 0.626 | 0.85 | 0.730 |
| 6 | R (1, 2, 3, 5) | 0.5 | 1.2 | 0 | 1.1 | 192.4 | 0.230 | 0.680 | 0.157 | 0.326 |

†Listed are the compositions per 1 mL nanoparticle suspensions.

TABLE 2C
Development of BTM nanoparticles.†

| | BRIJ 78 | TPGS | MIGLYOL 812 | Particle Size | |
|---|---|---|---|---|---|
| Trial | (mg) | (mg) | (mg) | (nm) | P.I. |
| 1 | 0.5 | 1.2 | 1.1 | 192.4 | 0.23 |
| 2 | 1.4 | 0.6 | 1 | 149 | 0.328 |
| 3 | 0.9 | 0.6 | 1.4 | 190 | 0.103 |
| 4 | 1.2 | 1.5 | 1.2 | 309.2 | 1.079 |

†Listed are the compositions per 1 mL nanoparticle suspensions.

### Example 4

### Development of G78 Nanoparticles by Sequential Simplex Optimization

A solid lipid, glyceryl tridodecanoate was selected as an alternative to lipid-based NPs. Glyceryl tridodecanoate was selected as a possibly direct replacement of E. Wax in the previously described E78 NPs due to the enhanced solubility of PX in glyceryl tridodecanoate. Thus, in this simplex optimization, there were two variables, glyceryl tridodecanoate (oil) and BRIJ 78 (surfactant). The initial simplex was directed by the MultiSimplex software based on the reference values of 4 mg for glyceryl tridodecanoate and 8 mg for BRIJ 78 in 2 mL water. Simplex optimization then proceeded as shown in Table 3. After 8 trials, the optimized composition reached nearly constant values in trials 9-11 of 1.6-1.9 mg for glyceryl tridodecanoate and 4-4.2 mg for BRIJ 78. Finally, trial 11 was identified as the most optimized composition because the composition gave the smallest particle size and the formulation could easily accommodate 150 μg/mL of paclitaxel.

TABLE 3
Simplex optimization for the development of G78 nanoparticles.†

| | | | Particle | | | |
|---|---|---|---|---|---|---|
| | | Glyceryl | Size | | Peak | |
| Trial | BRIJ 78 | Tridodecanoate | (nm) | P.I. | #^{a} | Membership^{b} |
| 1 | 3.5 | 1.5 | 157.2 | 0.3 | 2 | 0 |
| 2 | 4.5 | 1.8 | 153.5 | 0.36 | 1 | 4.77E−02 |
| 3 | 3.8 | 2.5 | 194.6 | 0.275 | 1 | 1.73E−02 |
| 4 | 4.8 | 2.8 | 195.3 | 0.25 | 2 | 0 |
| 5* | 3.8 | 1.8 | 161.9 | 0.258 | 1 | 0.138 |
| 6 | 4.5 | 1.1 | —c | — | — | — |
| 7 | 4.0 | 2.1 | 199 | 0.282 | 1 | 3.03E−03 |
| 8 | 3.3 | 2.2 | — | — | — | — |
| 9* | 4.2 | 1.9 | 161.3 | 0.274 | 1 | 0.125 |
| 10 | 4.0 | 1.6 | 156.4 | 0.325 | 1 | 8.38E−02 |
| 11* | 4.0 | 1.7 | 143.6 | 0.369 | 1 | 4.48E−02 |

†Listed are the compositions per 1 mL nanoparticle suspensions.
^{a}The peak numbers in nanoparticle distribution
^{b}Current membership has the same meaning with D value in desirability functions.
^{c}Unable to form nanoparticles based on this composition.

### Example 5

### Characterization of Nanoparticles

### Lyophilization of BTM and G78 Nanoparticles

The lyophilization of BTM NPs and PX BTM NPs in water alone resulted in the formation of dry white cakes that were rapidly rehydrated with water within less than 15 seconds to produce clear NP suspensions wherein the NPs showed complete retention of original physicochemical properties and in-vitro release properties (FIG. 2 and FIG. 6). In contrast, lyophilized G78 NPs or PX G78 NPs in the presence or absence of 5% lactose as a cryoprotectant could not be rehydrated in water and produced aggregates/agglomerates after rehydration.

### Particle Size and Zeta Potential

All tested nanoparticles had mean particle size diameters less than 200 nm with zeta potentials of about −6 mV regardless of PX entrapment. The entrapment of paclitaxel had no influence on the mean particle size of G78 and BTM nanoparticles (Table 4). Interestingly, rehydrated lyophilized NPs had smaller particle sizes for both blank BTM NPs and PX BTM NPs (FIG. 2).

### Drug Loading and Entrapment Efficiencies of Paclitaxel in Nanoparticles

HPLC analysis showed that the 7-epi isomer of PX was present at about 30% when PX was formulated in NPs in water. Further analysis showed that the epimerization occurred during preparation of the PX NPs (MacEachern-Keith et al., 1997). However, epimerization at C7 is reversible and can be prevented by forming PX NPs at slightly acidic pH (Tian and Stella, 2008). The 7-epi isomer of PX did not form when PX BTM NPs were prepared in 10% lactose (pH=5) or 50 mM sodium acetate buffer (pH=6). The slope of the standard curve for 7-epi PX was not statistically different from that for PX (data not shown). Thus, the standard curve for PX was used to determine the total PX concentration (PX plus 7-epi PX).

The entrapment efficiencies for PX G78 NPs and PX BTM NPs were 85% and 97.5%, respectively, as shown in Table 4. The mass balance of PX was 85.4±3.3% and 102.7±2.0% (mean±SD, n=3) for PX G78 NPs and PX BTM NPs, respectively. The results showed that paclitaxel was incorporated into nanoparticles at weight ratio of over 6% of the selected lipid core. Finally, rehydrated lyophilized PX BTM NPs showed 93.1% of entrapment efficiency, which was not statistically different to that of non-lyophilized PX BTM NPs (p>0.05).

TABLE 4
Physiochemical properties of PX G78, PX BTM, and lyo PX BTM nanoparticles (n = 3)

| | | | | | % Drug | |
|---|---|---|---|---|---|---|
| | Theoretical | Mean^{a} | | Zeta | Loading | % Drug |
| | Loading | Diameter | | Potential | (w/w, | Entrapment |
| Formulations | (μg/mL) | (nm) | P.I. | (mV) | drug/oil) | Efficiency |
| PX G78 NPs | 150 | 169.2 ± 8.1 | 0.302 ± 0.027 | −6.6 ± 2.6 | 7.5 | 85.4 ± 3.3 |
| PX BTM NPs | 150 | 190.5 ± 7.8 | 0.279 ± 0.054 | −5.9 ± 1.78 | 6 | 97.5 ± 2.6^{#} |
| Lyo PX BTM NPs | 150 | 130.0 ± 7.8 | 0.284 ± 0.042 | −5.1 ± 1.00 | 6 | 93.1 ± 4.1^{#} |

^{a}The data are presented as the mean of the mean particle size of nanoparticles in different batches ± SD (n = 3).
^{#}p > 0.05

### Physical Stability of Nanoparticles

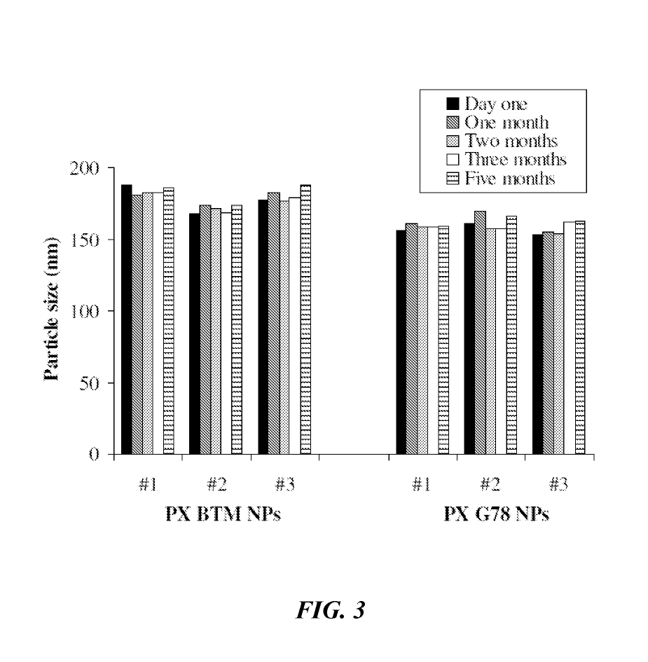

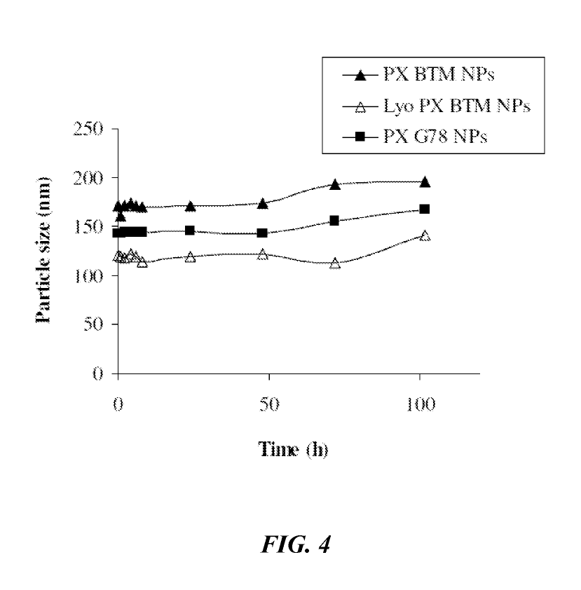

The physical stability of paclitaxel nanoparticles was evaluated by monitoring changes of particle sizes at 4° C. upon long-term storage, as well as short term stability at 37° C. in PBS to simulate physiological conditions. The particle sizes of G78 and BTM nanoparticles with or without paclitaxel did not significantly change at 4° C. for five months (FIG. 3). To test stability of nanoparticles in physiological condition, G78 NPs, BTM NPs and reconstituted lyophilized BTM NPs were incubated in PBS at 37° C. for 102 h. Particle sizes of PX-loaded NPs and blank NPs slightly increased after 72 h incubation. The data for PX-loaded NPs are shown in FIG. 4, whereas the data for blank NPs are not shown.

### Physical State of the Core Lipid in G78 Nanoparticles

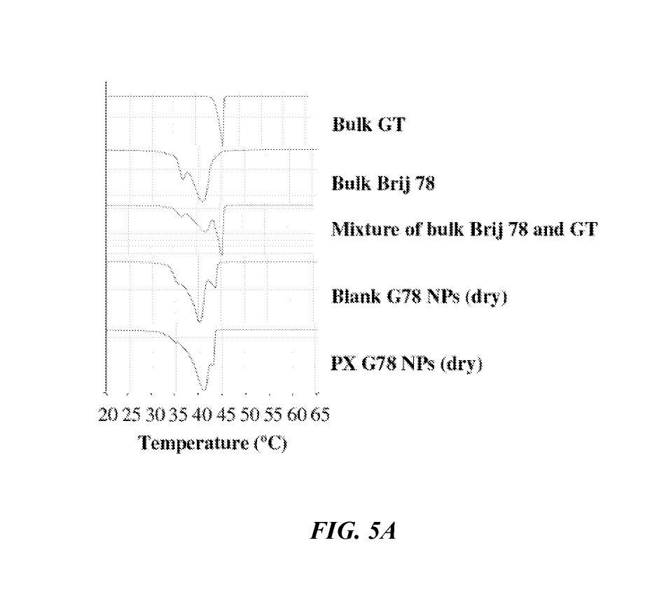

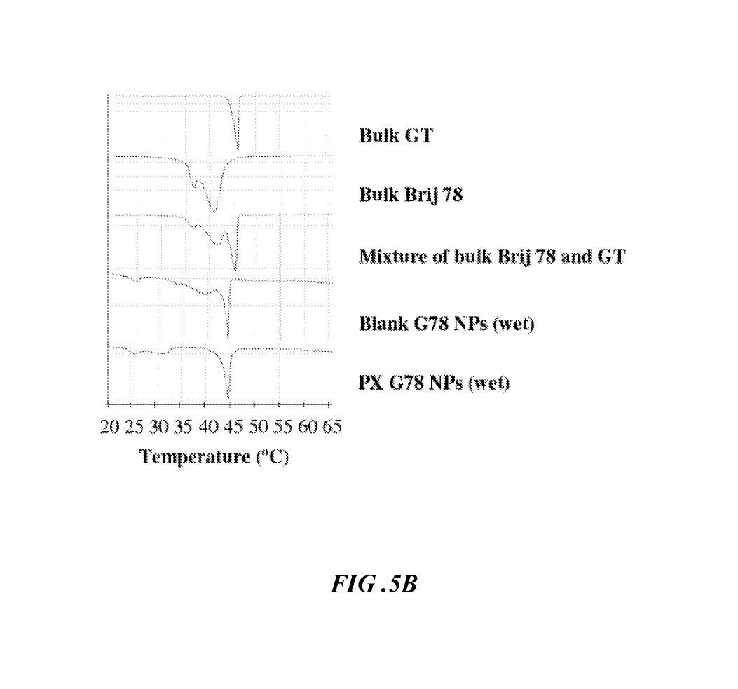

It has been reported that glyceryl tridodecanoate (also called ‘trilaurin’) existed as super-cooled melts rather than in a solid state in nanoparticles (Bunjes et al., 1996; Siekmann and Westesen, 1994). Thus, in the presently disclosed subject matter, DSC analysis was used to determine the physical state of glyceryl tridodecanoate in G78 nanoparticles. Bulk glyceryl tridodecanoate showed the melting peak at 46° C., while BRIJ 78 had two melting peaks at 35° C. and 40° C. The concentrated blank and PX G78 NPs clearly showed an endothermal peak at 43° C. (FIG. 5B). After drying of the NPs, two other peaks at 35° C. and 40° C. appeared for blank or PX G78 NPs (FIG. 5A). The endothermal peaks of BRIJ 78 intensified after drying suggesting that more BRIJ 78 existed in the solid state. The melting peak of glyceryl tridodecanoate in nanoparticles shifted to lower temperature and was broader compared to that of bulk material. However, the endothermic peak at 43° C. for glyceryl tridodecanoate indicated that glyceryl tridodecanoate retained a solid state in G78 nanoparticles.

In-Vitro Release of Paclitaxel from Nanoparticles

Paclitaxel has been reported to have aqueous solubility of 0.7-30 μg/mL. Therefore, to maintain sink conditions, PBS with 0.1% Tween 80 was used as the release medium for the in-vitro release studies of paclitaxel. The solubility of paclitaxel in release medium at room temperature was 10.8±0.3 μg/mL (mean±SD, n=3) as measured by HPLC. Thus, for the release studies, 800 μL of PX NPs containing 150 μg/mL of paclitaxel were placed into 40 mL of release medium. The cumulative release of paclitaxel from PX NPs is shown in FIG. 6. Free PX was released completely within 4 h. For all tested PX NPs, although the initial release rates were greater between 0 and 8 h, no initial burst of PX was observed. After 8 h, the release rates were much lower. The results showed that the mean cumulative release of PX after 72 h was 40%, 50% and 53% from PX G78 NPs, PX BTM NPs and reconstituted lyophilized PX BTM NPs, respectively. Mass balance analysis for PX G78 NPs, PX BTM NPs and lyophilized PX NPs showed that 79.2±8.6%, 98.3±24.2%, and 73.4±16.6% (mean±SD, n=4) of the PX was recovered, respectively. There were no other PX degradation peaks, except for 7-epi PX, observed by HPLC during the course of the studies. Moreover, lyophilized PX BTM NPs showed the same release profile as compared to PX BTM NPs (p>0.05 at each time point). Also, the particle sizes of all tested nanoparticles did not change significantly after 72 h.

### In Vitro Cytotoxicity Studies

The cytotoxicity of PX NPs was tested in human breast cancer MDA-MB-231 cells using the SRB assay (Table 5). PX NPs showed a clear dose-dependent cytotoxicity in MDA-MB-231 cells. There was no statistical significance in the IC50 values of PX BTM NPs and lyophilized PX BTM NPs compared to commercial TAXOL. However, the IC50 of PX G78 NPs had comparable but statistically different IC50 values compared to TAXOL. Blank NPs showed some cytotoxicity but only the paclitaxel equivalent dose of 617.3 nM and 354.6 nM of PX, which corresponds to a total NP concentration of 26.4 μg/mL and 15.1 μg/mL for blank G78 NPs and BTM NPs, respectively.

TABLE 5
IC50 Values of Paclitaxel Nanoparticles in MDA-MB-231 Cells at 48 h

| | | G78 NPs | | BTM NPs #1 | | BTM NPs #2^{a} | | Lyo BTM | NPs #2^{a} |
|---|---|---|---|---|---|---|---|---|---|
| Formulations | TAXOL | PX NPs* | BL NPs | PX NPs^{#} | BL NPs^{##} | PX NPs^{#} | BL NPs^{##} | PX NPs^{#} | BL NPs^{##} |
| IC50 (nM) | 7.2 ± 2.9 | 21.0 ± 1.5 | 617.3 ± 356 | 7.6 ± 1.2 | 354.6 ± 59.0 | 15.1 ± 6.8 | 342.7 ± 119.6 | 15.6 ± 10.6 | 256.1 ± 128.6 |

Data are presented as the mean ± SD of three independent experiments (N = 3) with triplicate (n = 3) measurements for each sample/concentration tested.
^{a}Lyo BTM NPs #2 were directly lyophilized from BTM NPs #2. Lyophilized powder was stored at 4° C. for overnight prior to completing the cytotoxicity studies.
^{#}p > 0.05 compared to IC50 of TAXOL
^{##}p > 0.05 within the group
*p < 0.05 compared to IC50 of TAXOL

As further described below, Sequential Simplex Optimization has been utilized to identify lipid nano-based paclitaxel formulations having useful attributes. Experimental design was performed combining Taguchi array and sequential simplex optimization. The combination of Taguchi array and sequential simplex optimization efficiently directed the design of paclitaxel nanoparticles. Two optimized paclitaxel nanoparticles (NPs) were obtained, G78 and BTM. G78 was found to be a solid lipid nanoparticle formulation, whereas BTM is thought to be a nanoemulsion particle or nanocapsule-based formulation. Both nanoparticles successfully entrapped paclitaxel at a final concentration of 150 μg/mL with particle sizes less than 200 nm and over 85% of entrapment efficiency. These novel paclitaxel nanoparticles were stable at 4° C. over five months and in PBS at 37° C. over 102 hours. Release of paclitaxel was slow and sustained without initial burst release. Cytotoxicity studies in MDA-MB-231 cancer cells showed that both nanoparticles have similar anticancer activities compared to TAXOL. Both formulations have been shown to overcome P-glycoprotein (P-gp) mediated resistance in human cancer cells via ATP depletion. PX BTM formulations are stable in suspension for at least 2 months at 4° C. Interestingly, it was surprisingly found that PX BTM NPs could be lyophilized without cryoprotectants. The lyophilized cakes comprised only of PX BTM NPs in water could be rapidly rehydrated with complete retention of original physicochemical properties, in-vitro release properties, and cytotoxicity profile. These nano-based formulations can be used for many different types of poorly-water soluble and insoluble drugs ideally for parenteral administration. Ideally, the BTM formulation can be lyophilized without cryoprotectants to retain all measured properties.

### Discussion

Paclitaxel (PX) is an important agent in the treatment of metastatic breast cancer. However, the optimal clinical use of paclitaxel is limited due to its poor aqueous solubility. Commercial paclitaxel formulation, TAXOL, is generally associated with hypersensitivity reactions that results from the excipient CREMOPHOR EL in TAXOL. To overcome the problems, numerous lipid-based and CREMOPHOR EL-free paclitaxel formulations have been investigated, such as liposomes (Zhang et al., 2005), solid lipid nanoparticles (Lee et al., 2007; van Vlerken et al., 2007), micelles (Sznitowska et al., 2008; Hassan et al., 2005), emulsions (Kan et al., 1999; Constantinides et al., 2000).

In the presently disclosed subject matter, two median chain triglycerides, glyceryl tridodecanoate and MIGLYOL 812, were used to investigate new lipid-based nanoparticles for paclitaxel. Relative to other candidate oil phases, these two oils have high solvation ability for PX. Glyceryl tridodecanoate has a relatively low melting point of 46° C., which theoretically facilitates the preparation of lower crystalline cores that can accommodate a greater concentration of drug (Manjunath et al., 2005). MIGLYOL 812, being a liquid, forms a reservoir-type drug delivery systems in which poorly water-soluble drugs remain dissolved inside the liquid oil core and consequently a high payload and reduced release profile can be achieved (Fresta et al., 1996; Mosqueira et al., 2000). The final optimized nanoparticles, G78 NPs and BTM NPs, successfully entrapped paclitaxel with high loading and entrapment efficiency (Table 4). However, the selection of these two alternative oil phases required the development of optimized NP formulations. To facilitate the development of optimized NP formulations, the presently disclosed subject matter uses a methodology that combined Taguchi array and sequential simplex optimization. The simplex is made of k+1 vertex. The response of the experiment in each vertex is ranked and the “worst” response is replaced by the new set of variables for the next experiment. To efficiently move the simplex, there should be limited “worst” responses in the starting simplex. As new excipients are encountered and no known compositions could be referred (Table 2A-2C), Taguchi array was first performed to explore and provide the framework of the starting simplex. The final optimization was then completed using sequential simplex optimization. Trial 6 in Table 4 identified a new nanoparticle formulation composed of the liquid oil MIGLYOL 812. After further optimization, new BTM nanoparticles were developed. For new compositions, which are referred to as E78 NPs (Table 3), the sequential simplex optimization was directly used for investigation of G78 NPs. The results for both PX NPs indicate that this new methodology combining Taguchi array and sequential simplex optimization could efficiently and effectively be used to identify optimized nanoparticles. To the knowledge of the inventors, this is the first report to use the combination of Taguchi array and sequential simplex optimization for the development of nanoparticles.

It was observed that the affinity or the solubility of the drug in the lipid core can predict the entrapment efficiency and release rate of the drug from the nanoparticles. The optimal PX BTM and G78 nanoparticles were reproducible with high drug loading, as well as slow release of PX achieving about 50% and 40% after 72 h, respectively (FIG. 6). The slow and sustained release of paclitaxel without burst release from PX BTM and PX G78 nanoparticles indicated that paclitaxel was likely not present at or near the surface of nanoparticles, but instead is present within the core of the NPs, which is consistent with the enhanced solvation ability of MIGLYOL 812 and glyceryl tridodecanoate for PX.

Moreover, entrapment of paclitaxel into nanoparticles did not change the sizes of nanoparticles. All PX NPs had particle sizes less than 200 nm, even after 102 h of incubation in PBS at 37° C. These data provide some evidence that the nanoparticles can have sufficient stability in the blood after intravenous injection (FIG. 4). Cytotoxicity studies showed that both PX G78 and BTM nanoparticles had the same or comparable anticancer activity compared to commercial TAXOL in human MDA-MB-231 breast cancer cells. Therefore, both of these identified PX NP formulation can be good candidates for ligand-mediated tumor-targeted delivery of PX.

Several studies have reported that glyceryl tridodecanoate is retained in lipid-based NPs in a super-cooled liquid state. If true, this semi-stable state of glyceryl tridodecanoate will likely affect the stability of nanoparticles due to the predicted phase transition of the super-cooled core to the crystalline phase. However, the presently disclosed subject matter using DSC analysis indicates that glyceryl tridodecanoate remained as a solid state in G78 NPs (FIG. 5), suggesting that the phenomenon of super-cooled glyceryl tridodecanoate in nanoparticles might be dependent on the process and compositions (i.e., surfactant) used to prepare the nanoparticles. Blank and PX G78 nanoparticles stored as liquid suspensions at 4° C. remained stable for several months and exhibited no change in particle size. Further, neither blank nor PX G78 nanoparticles showed a change in particle sizes after 102 h of incubation in PBS at 37° C., which indicates that the presently disclosed G78 nanoparticles, made with the lower melting GT, are not adversely affected by body temperature.

Without wishing to be bound by any one particular theory, it is thought that BTM NPs comprise a novel liquid reservoir or nanocapsule-type formulation. The liquid reservoir containing paclitaxel dissolved in MIGLYOL 812 is stabilized with the polymeric surfactants BRIJ 78 and TPGS. Higher drug loading of PX BTM nanoparticles demonstrates the advantage of this nanocapsule-type formulation as compared to the solid-core type G78 NP system. The BTM NPs were spontaneously formed after cooling from the warm o/w microemulsion precursors. Further, it is thought that the BTM NPs are nanocapsules and not nanoemulsions since nanoemulsions are non-equilibrium and thermodynamically unstable systems that cannot, by definition, form spontaneously without agitation or significant mechanical/shear mixing (Solans et al., 2005).

The following observation was made serendipitously during the course of the present studies. In one attempt to concentrate NP formulations to analyze for entrapped PX, NPs were lyophilized in water. The BTM NP formulations produced uniform white cakes that could be rapidly rehydrated with complete retention of the original physicochemical properties, in-vitro release properties, and cytotoxicity profile. The inventors' experience, as well as that of others, suggests that it is often difficult to freeze-dry colloidal suspensions in the presence of cryoprotectants. To the inventors' knowledge, there are few or no reports of the successful lyophilization of colloidal suspensions without the use of a cryoprotectant that protects the nanoparticles from the stresses of the freezing and thawing process. Moreover, the lyophilization of nanoemulsion particles or nanocapsules is thought to be even more challenging due to the existence of the very thin and fragile lipid envelope that typically cannot withstand the mechanical stress of freezing (Schaffazick et al., 2003; Abdelwahed et al., 2006). Even in the presence of cryoprotectants, an increase of particle size is likely to occur (Heurtault et al., 2002). In the presently disclosed subject matter, the optimal BTM nanoparticles were successfully lyophilized without cryoprotectants. The non-collapsed uniform cakes of PX BTM NPs in water alone were rehydrated and spontaneously produced particle sizes that were, in fact, slightly smaller than the original particle sizes. In addition, there was complete retention of the in-vitro release properties and cytotoxicity profile.

In conclusion, the combination of Taguchi array and sequential simplex optimization efficiently guided the development and optimization of lipid-based nanoparticulate formulation for paclitaxel. Injectable paclitaxel nanoparticles, PX G78 NPs and PX BTM NPs, were successfully prepared via a warm o/w microemulsion precursor engineering method. Both paclitaxel nanoparticles were physically stable at 4° C. over five months, and PX BTM could be lyophilized without cryoprotectants. PX G78 and PX BTM nanoparticles showed comparable or the same anticancer activity compared to TAXOL in MDA-MB-231 breast cancer cells. Therefore, the presently disclosed paclitaxel-loaded nanoparticles can be used for ligand-mediated tumor-targeted delivery of paclitaxel, for example, after intravenous injection.

### Example 6

### Preparation of Nanocapsule Formulations without Heating

A nanoemulsion or nanocapsule formulation also was made without heating. Briefly, 2.5 mg of MIGLYOL 812, 1.5 mg of TPGS and 3.5 mg of BRIJ 78 were mixed/dissolved in ethanol. The ethanol was evaporated and 1 mL water was added. The system was mixed overnight at room temperature. The system was slightly turbid the next day. Particle size was 192 nm with a polydispersity index of 0.134.

### Example 7

### Preparation of Long-Circulating Nanoemulsion Particles or Nanocapsules

A one (1) mL suspension was prepared from warm o/w microemulsion precursors by adding 2.5 mg of MIGLYOL 812, 1.5 mg of TPGS and 3 mg of BRIJ 78 to a glass vial and heating to 65° C. 975 microliters of filtered and deionized (D.I.) water pre-heated at 65° C. was added into the mixture of melted oils and surfactants. After 15 min of mixing, 25 microliters of an 8 mg BRIJ 700/mL stock solution was added to the warm mixture and mixed for an additional 10 min. The mixture was then cooled to room temperature and stirred for 5 hr. BRIJ 700, also known as Steareth 100, has a PEG moiety (Mw of PEG about 4400) and is added to the formulation to form sterically stabilized nanoparticles to make the formulation long circulating in the blood.

### Example 8

### Preparation of Concentrated Paclitaxel Nanocapsule Formulations

Paclitaxel nanocapsules were made more concentrated during the manufacturing process by increasing the mass of excipients in the formulation but keeping the volume of water constant at 1 mL.

### 3× Concentrated Paclitaxel Nanocapsules

450 μg paclitaxel, 7.5 mg of MIGLYOL 812, 4.5 mg of TPGS and 10.5 mg of BRIJ 78 were mixed at 65° C., and then 1 mL water was added. After 20 min mixing at 65° C., the system was cooled to room temperature. The concentration of paclitaxel in the nanocapsule suspension before and after filtration through a 0.2 micron filter was 518.1+/−3.3 μg/mL and 504.5+/−1 μg/mL, respectively.

### 4× Concentrated Paclitaxel Nanocapsules

600 μg paclitaxel, 10.0 mg of MIGLYOL 812, 6.0 mg of TPGS and 14.0 mg of BRIJ 78 were mixed at 65° C., and then 1 mL water was added. After 20 min mixing at 65° C., the system was cooled to room temperature. The concentration of paclitaxel in the nanocapsule suspension before and after filtration through a 0.2 micron filter was 671.3+/−1.6 μg/mL and 689.6+/−1.5 μg/mL, respectively.

### 5× Concentrated Paclitaxel Nanocapsules

750 μg paclitaxel, 12.5 mg of MIGLYOL 812, 7.5 mg of TPGS and 17.5 mg of BRIJ 78 were mixed at 65° C., and then 1 mL water was added. After 20 min mixing at 65° C., the system was cooled to room temperature. The concentration of paclitaxel in the nanocapsule suspension before and after filtration through a 0.2 micron filter was 794.6+/−1.8 μg/mL and 773.7+/−1.1 μg/mL, respectively.

### Example 9

### Methods of BTM Formulations to Overcome P-gp Mediated Resistance in Human Cancer Cells

The following data are the IC50 values in three different human cancer cells comparing paclitaxel (PX) BTM, Blank (placebo) BTM, and TAXOL. The results presented in Table 6 show that PX BTM leads to a log-reduction in the IC50 as compared to TAXOL in a P-gp-overexpressing human cancer cell line.

TABLE 6
IC₅₀ values in Human Cancer Cells^{†}

| | P-gp | IC50 (μM) | | |
|---|---|---|---|---|
| Cell lines | expression | TAXOL | PX BTM | Blank BTM |
| MDA-MB-231 | − | 7.23 ± 2.89 | 7.63 ± 1.15 | 355 ± 59.0 |
| OVCAR-8 | − | 7.70 ± 1.82 | 11.3 ± 9.07 | 252 ± 61.3 |
| NCI/ADR-RES | + | 3814 ± 721 | 391 ± 81.7 | 548 ± 111 |

^{†}Cytotoxicity studies were carried out using Sulfrhodamine B Assay. All groups included three independent experiments (N = 3) with triplicates (n = 3) for each experiment.

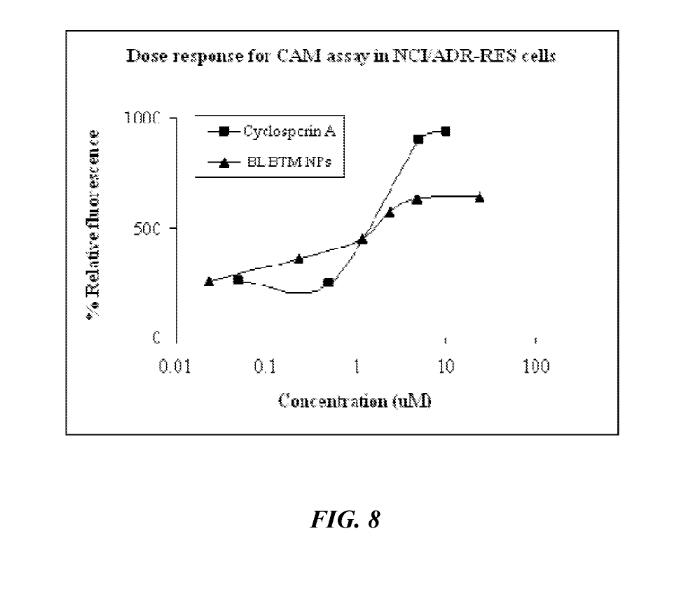

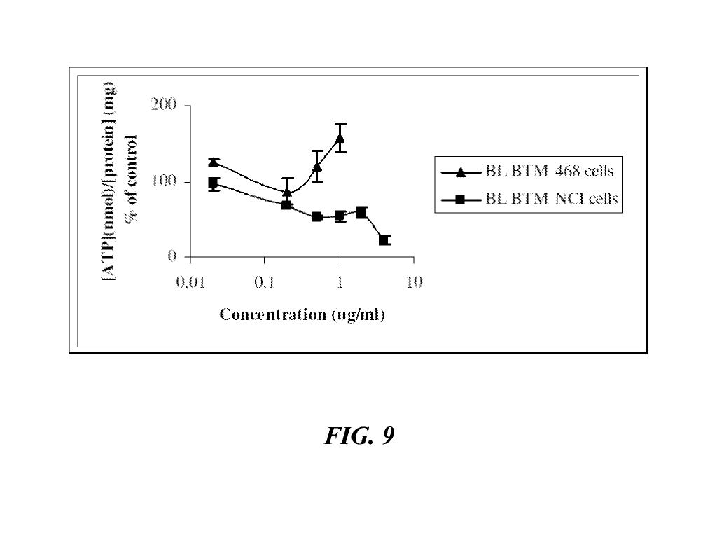

To test effects of blank BTM nanocapsules on P-gp, a Calcein AM assay was performed. Calcein AM is a substrate of P-gp and is non-fluorescent. Once entering cells, calcein AM is irreversibly converted by cytosolic esterases to calcein, a non-permeable and fluorescent molecule. Thus, the increased intracellular fluorescence of calcein when P-gp-overexpressing cells were exposed to lipid-based NPs indicates the inhibition of P-gp function. In NCI/ADR-RES (resistant) cells, blank BTM nanocapsules led to a linear increase in calcein fluorescence over 1 hr (FIG. 7). Moreover, the fluorescence caused by intracellular calcein significantly increased in a dose-dependent manner when cells were treated with various doses of blank BTM nanocapsules (equivalent concentrations of PX) (FIG. 8). In stark contrast, no treatments led to increased intracellular fluorescence compared to calcein AM alone in the sensitive MDA-MB468 cells (data not shown). Under all conditions tested, the trypan blue assay confirmed that there was no significant loss of cell membrane integrity in NCI/ADR-RES and MDA-MB-468 cells. BTM nanocapsules also were found to deplete ATP in P-pg resistant NCI cells in a dose dependent manner; however, they did not deplete ATP in non P-gp-overexpressing MDA-MB-468 cells (FIG. 9).

### Example 10

### Coating His-Tagged Proteins on BTM NPs

BTM NPs having Nickel on the surface were prepared using 1,2-di-(9Z-octadecenoyl)-sn-glycero-3-[(N-(5-amino-1-carboxypentyl)imidodiacetic acid)succinyl] (nickel salt) (DGS-NTA-Ni). BRIJ 78, Vitamin E TPGS and MIGLYOL 812 were weighed in a 7-mL scintillation vial. DGS-NTA-Ni was added as a 10 mg/mL solution in chloroform. The weight (mg/mL NPs) of each component is provided in Table 7. The vial was transferred to a water bath at 70° C. Preheated water was added to the vial and stirred for 30 min. The vial was cooled to room temperature (RT). The NPs were passed through a sepharose CL-4B column to separate unincorporated components. NPs of size 187.8±0.32 nm and zeta potential of −11.3±7.1 were obtained. Ni content of the NPs was determined using ICP-MS (Inductively Coupled Plasma-Mass Spectrometry). The NPs had 145.6±19.53 ng Ni/mg NPs.

TABLE 7
Components for Coating his-tagged proteins on BTM NPs

| Components | Weight (mg/mL NPs) |
|---|---|
| BRIJ 78 | 3.5 |
| Vit E TPGS | 1.5 |
| MIGLYOL 812 | 2.5 |
| DGS-NTA-Ni | 12.5 μL |

Binding of his-GFP (Green Fluorescent Protein) to the NPs was evaluated by incubating his-GFP with the BTM Ni-NP suspension at 4° C. overnight. Unbound GFP was separated using a sepharose CL-4B column. 480 μg NPs could completely bind 1 μg GFP.

To use the BTM NPs as a vaccine delivery system, the binding of the HIV protein his-P41 to BTM Ni-NPs was performed. The particles size of the protein bound NPs was 177.6±0.53 nm. Balb/c mice were immunized on day 0 and 14 with 0.1 mL of BTM Ni-NPs coated with his-P41. The dose levels for his-P41 were 1 μg, 0.5 μg, or 0.1 μg and the corresponding dose of NPs was 480 μg, 240 μg, or 48 μg, respectively. On day 28, mice were bled by cardiac puncture and sera were collected and analyzed for total IgG, IgG1, and IgG2a by ELISA.

### Example 11

### In Vivo Anticancer Efficacy Study #1

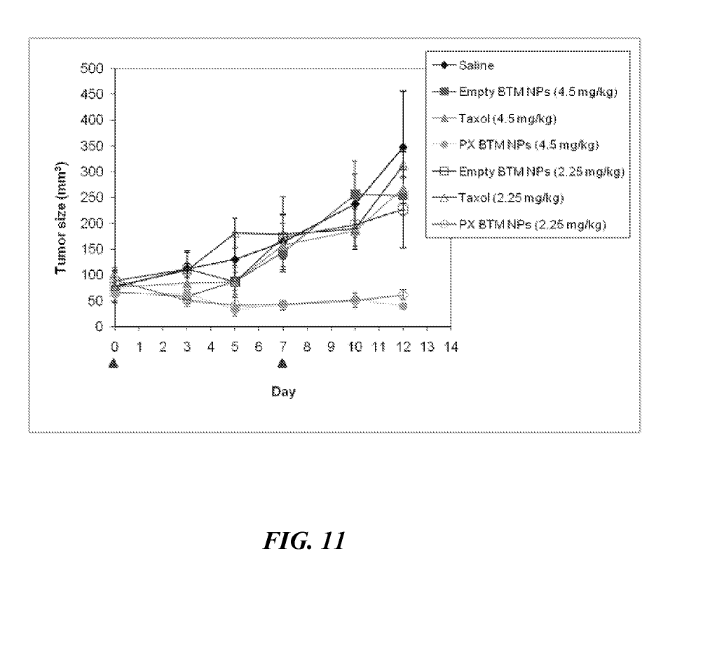

In vivo anticancer efficacy study #1 used pegylated PX BTM NPs in resistant mouse NCI/ADR-RES xenografts. On Day (−7), 18-19 g female nude mice received 4×10⁶ cells by s.c. injection. Mice (n=4/group) were dosed i.v. with PX (4.5 or 2.25 mg/kg) by tail vein injection on day 0 and 7. The corresponding nanoparticle dose was 210 or 105 mg NPs/kg, respectively. Data are presented in FIG. 11 as the mean±SD.

In this study, tumor volume increased with control, TAXOL, and blank BTM NPs administration at the two paclitaxel or paclitaxel-equivalent doses tested. In comparison, a marked anticancer effect of the pegylated paclitaxel BTM nanoparticles was clearly observed (FIG. 11). The tumor volume in the two tested pegylated paclitaxel BTM nanoparticles groups exhibited almost no change during the course of the study. A statistically significant difference of pegylated paclitaxel BTM nanoparticles from all other treatments was observed from day 5 and continued to the end of the study. Blank BTM nanoparticles did not show any clinical signs of toxicity even at the highest dose of 210 mg nanoparticles/kg.

### Example 12

### In Vivo Anticancer Efficacy Study #2

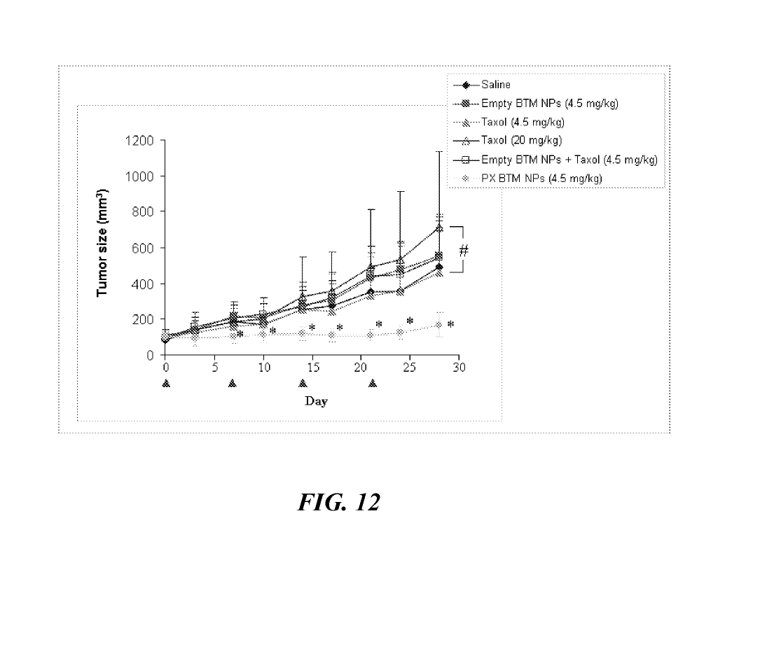

In-vivo anticancer efficacy study #2 used pegylated PX BTM NPs in resistant mouse NCI/ADR-RES xenografts. Female nude mice received 4×10⁶ cells by s.c. injection. Mice (n=6/group) were dosed i.v. with PX (4.5 mg/kg) by tail vein injection on day 0, 7, 14, and 21 in the form of either TAXOL, PX BTM NPs, or TAXOL spiked in blank BTM NPs. TAXOL (20 mg/kg) near or at the maximum tolerated dose as well as blank NPs with a dose of NPs equal to that of PX BTM NPs were added as controls. The corresponding nanoparticle dose was 210 mg NPs/kg, respectively. Data are presented in FIG. 12 as the mean±SD.

### Example 13

### Retreatment of Selected Groups in In Vivo Anticancer Efficacy Study #2

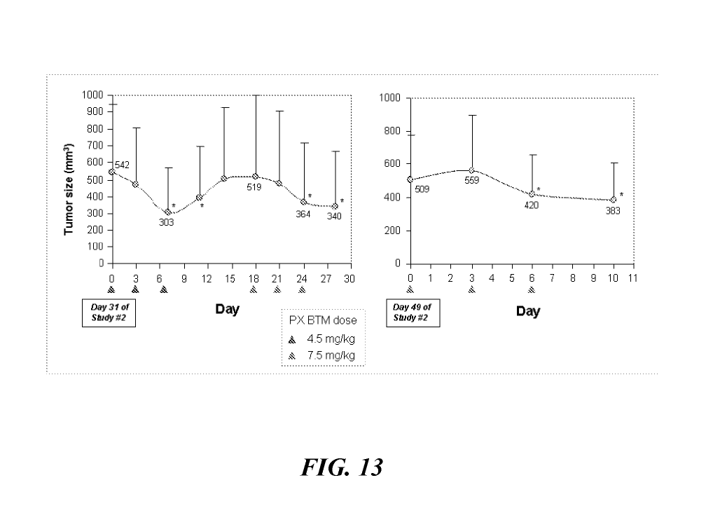

Selected groups from study #2 described immediately hereinabove (shown in FIG. 12, were retreated to determine if the presently disclosed NPs could salvage TAXOL-failed mice. The left panel of FIG. 13 shows TAXOL-failed mice from efficacy study #2 that were combined and then treated with PX BTM NPs. Doses and dosing schedule of PX BTM NPs to the TAXOL-failed mice are shown in the legend of FIG. 13. As depicted in FIG. 13, the treatment of TAXOL-failed mice with PX BTM NPs significantly (p<0.05) reduced tumor sizes demonstrating efficacy in treating TAXOL-failed mice. In the right panel of FIG. 13, previously PX BTM NP-treated mice were retreated with PX BTM NPs at the doses and dosing schedule shown in the legend. The retreatment significantly (p<0.05) reduced tumor sizes demonstrating that retreatment with PX BTM NPs provided efficacy. Data are presented in FIG. 13 as the mean±SD.

### Example 14

### Gd-MRI Imaging of BTM-DTPA-Gd Nanoparticles

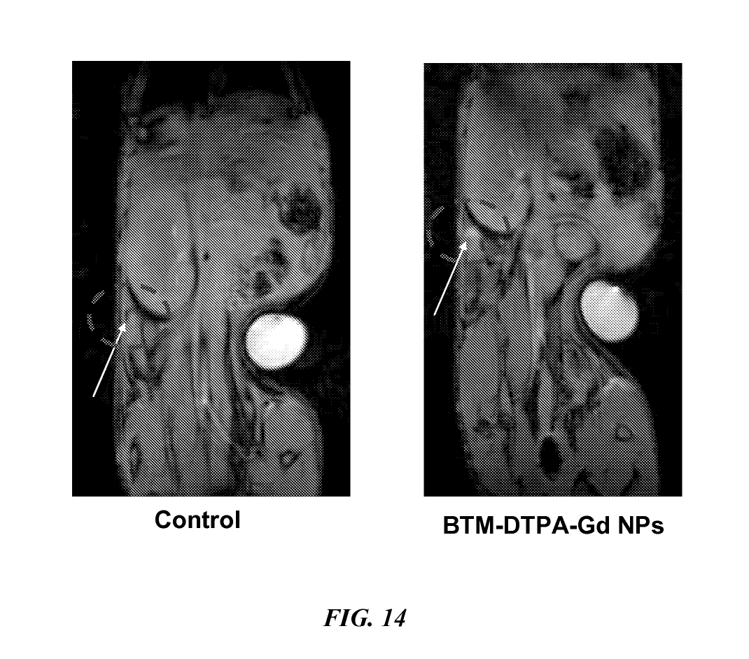

BTM NPs were prepared with accessible DTPA on the surface of the NPs using methods described by Zhu et al., “Nanotemplate-engineered nanoparticles containing gadolinium for magnetic resonance imaging of tumors,” Invest Radiol. 43(2):129-40 (2008). The BTM-DTPA-Gd NPs were injected into nude mice bearing A549 tumors. Five hours after injection, MRI images were obtained using a 9.4T Micro-MRI. The results showed that the BTM-DTPA-Gd NPs provided positive tumor contrast (FIG. 14, panel at right) were control (FIG. 14, panel on left).

### Example 15

### Preparation of Nanocapsules at Room Temperature

Nanocapsules were prepared by adding to a glass vial, 5 mg MIGLYOL 612 and 5 mg vitamin E TPGS. The excipients were dissolved with 100 mL of ethanol and mixed, and the ethanol was then evaporated with a stream of nitrogen gas. Two (2) mL of water was then added to the vial while stirring. The mixture was stirred at room temperature for 20 minutes. The formed nanocapsules had a mean size of 224.4±2.34 nm and a P.I. of 0.010±0.023 with a unimodal distribution. SDP intensity analysis showed a mean size of 228.2±35.46 nm. MIGLYOL 612, or glyceryl trihexanoate, is a shorter chain molecule and can function as both an oil phase and surfactant in this formulation. This phenomenon is referred to as “self-emulsification.”

### REFERENCES

The following references, to the extent that they provide exemplary procedural or other details supplementary to those set forth herein, are specifically incorporated herein by reference. Further, all publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
U.S. Pat. No. 4,870,287;
U.S. Pat. No. 5,399,363;
U.S. Pat. No. 5,466,468;
U.S. Pat. No. 5,543,158;
U.S. Pat. No. 5,641,515;
U.S. Pat. No. 5,686,072;
U.S. Pat. No. 5,725,871;
U.S. Pat. No. 5,756,353;
U.S. Pat. No. 5,760,395;
U.S. Pat. No. 5,780,045;
U.S. Pat. No. 5,804,212;
U.S. Pat. No. 6,537,514;
U.S. Pat. No. 6,613,308;
U.S. Pat. No. 7,153,525;
U.S. Pat. No. 4,664,911;
U.S. Pat. No. 4,792,447;
U.S. Pat. No. 5,045,451;
U.S. Pat. No. 5,578,706;
U.S. Pat. No. 5,767,072;
Al Khouri et al., Pharmaceutica Acta Helvetiae, 61(10-11):274-81, 1986;
Alonso et al., Boll. Chim. Farm., 139(3): 114-9, 2000;
Alonso et al., Ciencia y Tecnologia Pharmaceutica, 9(4): 165-172, 1999;
Anton et al., J. Control Rel., 128(3):185-99, 2008;
Attwood, D., “Microemulsions,” In: Colloidal Drug Delivery Systems, Kreuter (ed.), 31-71, 1994.
Bhargava et al. Gen. Pharmacol., 18(3):337-40, 1987;
Bhaysar et al., J. Control Rel., 110(2):422-430, 2006;
Bummer, Crit. Rev. Ther. Drug Carrier Syst., 21(1):1-20, 2004;
Bunjes et al., Int. J. Pharm., 129:159-173, 1996;
Constantinides et al., Pharm. Res., 17(2):175-182, 2000;
Constantinides, Pharm. Res., 12(11): 156 1-72, 1995;
Culver et al., Science, 256(5063):1550-1552, 1992;
DE 19852245;
Derringer and Suich, J. Qual. Tech., 12:214-219, 1980;
Dhanikula et al., Biomaterials, 28(6): 1248-1257, 2007;
Dong and Mumper, Drug Dev. Ind. Pharm., 32(8):973-980, 2006;
El-Laithy, Curr. Drug Deliv., 5(3):170-176 2008;
EP 0711556A1;
EP 0711557A1;
EP 0865792;
Fresta et al., Biomaterials, 17(8):751-758, 1996;
Gabrielsson et al., J. Chemometrics, 16:141-160, 2002;
Gohel and Amin, J. Control Rel., 51(2-3):115-122, 1998;
Goodman & Gilman's “The Pharmacological Basis of Therapeutics;
Hassan et al., Cancer Chemother. Pharmacol., 55(1):47-54, 2005;
Heurtault et al., Pharm. Res., 19(6):875-880, 2002;
Hobbs et al., Proc. Natl. Acad. Sci. USA, 95(8):4607-4612, 1998;
Hubert et al., Congr. Int. Technol. Pharm., 4(5): 15-22, 1989;
Jeong et al., J. Microencapsul., 22(6):593-601, 2005;
Kan et al., J. Control Rel., 58(3):271-278, 1999;
Kang et al., Int. J. Pharm., 274(1-2):65-73, 2004;
Koziara et al., J. Control Rel., 112(3):3 12-319, 2006;
Koziara et al., J. Control Rel., 99(2):259-269. 2004;
Koziara et al., Pharm. Res., 22(11): 1821-1828, 2005;
Lee et al., Biomaterials, 28(12):2137-2146, 2007;
Liu et al., Drug Dev. Ind. Pharm., 34(5):465-71, 2008;
MacEachern-Keith et al., Anal. Chem., 69:72-77, 1997;
Maeda and Sawa, J. Control Rel., 74(1-3):47-61, 2001;
Manjunath et al., Exp. Clin. Pharmacol., 27(2): 127-144, 2005;
Mathew et al., J. Med. Chem., 35(1):145-151, 1992;
Montasser et al., Polymer Bulletin (Berlin, Germany), 50(3): 169-174, 2003;
Mosqueira et al., J. Pharm. Sci., 89(5):614-626, 2000;
Muggia, Clin. Cancer Res., 5(1):7-8, 1997;
Muller et al., Int. J. Pharm., 242(1-2):121-128, 2002;
Oyewumi et al., Drug Dev. Ind. Pharm., 28(3):317-328, 2002;
Papazisis et al., J. Immunol. Methods, 208:151-158, 1997;
Pasqualini et al., Trends Mol. Med., 8(12):563-571, 2002;
PCT Appln. WO 9904766;
Physicians Desk Reference;
Remington: The Science and Practice of Pharmacy, 21st Ed., University of the Sciences in Philadelphia;
Remington's Pharmaceutical Sciences” 15^{th} Ed., 1035-1038 and 1570-1580, 1990;
Remington's Pharmaceutical Sciences, 18^{th} Ed., Mack Printing Company, 1990;
Roy, In: Design of Experiments Using the Taguchi Approach, John Wiley & Sons, Inc., NY, 2001;
Sadurni et al., Eur. J. Pharm. Sci., 26(5):438-445, 2005;
Sarker, Curr. Drug Deliv., 2(4):297-310, 2005;
Schaffazick et al., Eur. J. Pharm. Biopharm., 56(3):(2006) 178-188, 2006;
Siekmann and Westesen, Colloids Surf B. Biointerfaces, 3:159-175, 1994;
Solans et al., Curr. Opin. Colloid Interface Sci., 10:102-110, 2005;
Streitwieser and Heathcock, In: Introduction to Organic Chemistry, 3^{rd}. Ed., 465-467, 1985;
Swindell and Krauss, J. Med. Chem., 34:1176-1184, 1991;
Sznitowska et al., Chem. Pharm. Bull., 56(1):70-74, 2008;
Takenaga, M. et al., J. Controlled Release 52(1-2):81-87, 1998;
The Merck Index, Eleventh Edition;
Tian and Stella, J. Pharm. Sci., 97(3):1224-1235, 2008;
Traul et al., Food Chem. Toxicol., 38(1):79-98, 2000;
van Vlerken et al., Cancer Res., 67(10):4843-4850, 2007;
Walters et al., In: Sequential Simplex Optimization, CRC Press Inc., FL, 1991;
Weiss et al., J. Clin. Oncol., 8(7):1263-1268, 1990;
Zhang et al., Eur. J. Pharm. Biopharm., 59(1):177-187, 2005; and
Zhu et al., Invest Radiol. 43(2):129-40 (2008).

All of the compositions and methods disclosed and claimed herein can be made and executed without undue experimentation in light of the present disclosure. While the compositions and methods of this invention have been described in terms of preferred embodiments, it will be apparent to those of skill in the art that variations can be applied to the compositions and methods and in the steps or in the sequence of steps of the method described herein without departing from the concept, spirit and scope of the invention. More specifically, it will be apparent that certain agents that are both chemically and physiologically related can be substituted for the agents described herein while the same or similar results would be achieved. All such similar substitutes and modifications apparent to those skilled in the art are deemed to be within the spirit, scope and concept of the invention as defined by the appended claims and equivalents thereof.

That which is claimed:

1. A nanocapsule or nanoemulsion particle comprising a pharmaceutically acceptable liquid oil phase, a surfactant, and optionally a co-surfactant; wherein the liquid oil phase comprises one or more compounds having the structure: 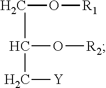
wherein:
Y is selected from the group consisting of H and —O—R₃;
R₁, R₂, and R₃ are each independently selected from the group consisting of 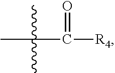
and H; wherein if R₁ is H and R₂ is H, then Y is not H and R₃ is not H;
R₄ is selected from the group consisting of C₁-C₂₅ alkyl, C₁-C₂₅ alkenyl, C₁-C₂₅alkylyl, and 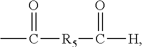
wherein R₅ is —(CH₂)ₓ—, and wherein x is an integer from 1 to 12.

2. The nanocapsule or nanoemulsion particle of claim 1, wherein R₁ or R₂ is 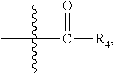
and wherein R₄ is selected from the group consisting of C₄-C₁₈ alkyl, C₈-C₂₅ alkenyl, and C₈-C₂₅ alkylyl.

3. The nanocapsule or nanoemulsion particle of claim 2, wherein R₄ is —(CH₂)_{y}—, and wherein y is an integer from 8 to 10.

4. The nanocapsule or nanoemulsion particle of claim 1, wherein the liquid oil phase comprises a component selected from the group consisting of an esterified caprylic fatty acid, an esterified capric fatty acid, an esterified glycerin, and an esterified propylene glycol.

5. The nanocapsule or nanoemulsion particle of claim 4, wherein the liquid oil phase comprises a component selected from the group consisting of triglyceryl monoleate, glyceryl monostearate, glyceryl trihexanoate, a medium chain monoglyceride or diglyceride, glyceryl monocaprate, glyceryl monocaprylate, decaglycerol decaoleate, triglycerol monooleate, triglycerol monostearate, a polyglycerol ester of a mixed fatty acid, hexaglycerol dioleate, a decaglycerol mono- or dioleate, propylene glycol dicaprate, propylene glycol dicaprylate/dicaprate, glyceryl tricaprylate/caprate, glyceryl tricaprylate/caprate/laurate, glyceryl tricaprylate/caprate, triacetin, propylene glycol di-(2-ethylhexanoate), glyceryl tricaprylate/caprate/linoleate, glyceryl tricaprate, glyceryl tricaprylate, and glyceryl triundecanoate.

6. The nanocapsule or nanoemulsion particle of claim 4, wherein the liquid oil phase comprises a naturally derived liquid oil.

7. The nanocapsule or nanoemulsion particle of claim 6, wherein the naturally derived liquid oil is selected from the group consisting of corn oil, coconut oil, sunflowerseed oil, vegetable oil, cottonseed oil, mineral oil, peanut oil, sesame oil, soybean oil, and olive oil.

8. The nanocapsule or nanoemulsion particle of claim 1, wherein at least one of the surfactant and the co-surfactant has a hydrophilic-lipophilic balance (HLB) of from about 6 to about 20.

9. The nanocapsule or nanoemulsion particle of claim 8, wherein at least one of the surfactant and the co-surfactant has a hydrophilic-lipophilic balance (HLB) of from about 8 to about 18.

10. The nanocapsule or nanoemulsion particle of claim 1, wherein at least one of the surfactant and co-surfactant is selected from the group consisting of a polyoxyethylene alkyl ether, a polyoxyethylene sorbitan fatty acid ester, a phospholipid, a polyoxyethylene stearate, a fatty alcohol, and hexadecyltrimethyl-ammonium bromide.

11. The nanocapsule or nanoemulsion particle of claim 1, wherein at least one of the surfactant and the co-surfactant is selected from the group consisting of d-alpha-tocopheryl polyethylene glycol 1000 succinate (TPGS) and polyoxyethylene 20-stearyl ether.

12. The nanocapsule or nanoemulsion particle of claim 1, wherein the surfactant is polyoxyethylene 20-stearyl ether, and wherein the co-surfactant is d-alpha-tocopheryl polyethylene glycol 1000 succinate (TPGS).

13. The nanocapsule or nanoemulsion particle of claim 1, wherein the liquid oil phase comprises a caprylic/capric triglyceride; wherein the surfactant is d-alpha-tocopheryl polyethylene glycol 1000 succinate (TPGS); and wherein the co-surfactant is polyoxyethylene 20-stearyl ether.

14. The nanocapsule or nanoemulsion particle of claim 13, wherein the nanocapsule or nanoemulsion particle comprises a ratio of caprylic/capric triglyceride:TPGS:polyoxyethylene 20-stearyl ether of about 1-3:1-3:1-5 (w:w:w).

15. The nanocapsule or nanoemulsion particle of claim 1, wherein the nanocapsule or nanoemulsion particle further comprises at least one bioactive agent.

16. The nanocapsule or nanoemulsion particle of claim 15, wherein the at least one bioactive agent is a substantially water-insoluble or a lipophilic drug and wherein the at least one bioactive agent is substantially comprised in the liquid oil core of the nanocapsule or the nanoemulsion particle.

17. The nanocapsule or nanoemulsion particle of claim 16, wherein the bioactive agent is selected from the group consisting of a small molecule, a therapeutic agent, an anti-viral agent, a bacteriostatic or anti-bacterial agent, an anti-fungal agent, a cell-targeting ligand, a peptide, a protein, a carbohydrate, a diagnostic agent, and a viral or bacterial protein capable of eliciting a humoral or cellular-based immune response.

18. The nanocapsule or nanoemulsion particle of claim 17, wherein the therapeutic agent is a chemotherapeutic agent.

19. The nanocapsule or nanoemulsion particle of claim 18, wherein the chemotherapeutic agent is paclitaxel.

20. The nanocapsule or nanoemulsion particle of claim 15, wherein the nanocapsule or nanoemulsion particle can be lyophilized and subsequently rehydrated without substantially affecting a potency of the nanocapsule or nanoemulsion particle after re-hydration, as compared to the potency of the nanocapsule or nanoemulsion particle prior to the lyophilization.

21. The nanocapsule or nanoemulsion particle of claim 20, wherein the bioactive agent is a chemotherapeutic agent, and wherein the potency comprises at least one of the in vitro and the in vivo cytotoxicity of the nanocapsule or nanoemulsion particle.

22. The nanocapsule or nanoemulsion particle of claim 15, wherein the bioactive agent is conjugated to the nanocapsule or the nanoemulsion particle.

23. The nanocapsule or nanoemulsion particle of claim 22, wherein the bioactive agent comprises an imaging agent.

24. The nanocapsule or nanoemulsion particle of claim 23, wherein the imaging agent is a magnetic resonance image (MRI) enhancement agent.

25. The nanocapsule or nanoemulsion particle of claim 24, wherein the MRI enhancement agent comprises a gadolinium-diethylenetriaminepentaacetic acid complex.

26. The nanocapsule or nanoemulsion particle of claim 1, wherein the surfactant is conjugated to a moiety selected from the group consisting of polyethylene glycol and polyoxyethylene.

27. The nanocapsule or nanoemulsion particle of claim 1, wherein the nanocapsule or nanoemulsion particle further comprises a cryoprotectant.

28. The nanocapsule or nanoemulsion particle of claim 1, wherein the nanocapsule or nanoemulsion particle is a lyophilized nanocapsule or nanoemulsion particle.

29. The nanocapsule or nanoemulsion particle of claim 1, further comprising a plurality of the nanocapsules or nanoemulsion particles, and wherein substantially all of the plurality of the nanocapsules or nanoemulsion particles has a particle size diameter less than about 300 nm.

30. A pharmaceutically acceptable formulation comprising the nanocapsule or nanoemulsion particle of claim 1.

31. The pharmaceutically acceptable formulation of claim 30, wherein the formulation is formulated for an administration route selected from the group consisting of parenteral, topical, rectal, oral, inhalation, intranasal, transdermal, and buccal administration.

32. A method of treating a disease comprising administering to a subject in need of treatment thereof, one or more nanocapsules or nanoemulsion particles comprising a pharmaceutically acceptable liquid oil phase, a surfactant, and optionally a co-surfactant; wherein the liquid oil phase comprises one or more compounds having the structure: 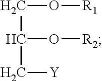
wherein:
Y is selected from the group consisting of H and —O—R₃;
R₁, R₂, and R₃ are each independently selected from the group consisting of 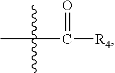
and H; wherein if R₁ is H and R₂ is H, then Y is not H and R₃ is not H;
R₄ is selected from the group consisting of C₁-C₂₅ alkyl, C₁-C₂₅ alkenyl, C₁-C₂₅alkylyl, and 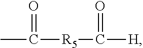
wherein R₅ is —(CH₂)ₓ—, and wherein x is an integer from 1 to 12; and
wherein the nanocapsules or nanoemulsion particles comprise at least one bioactive agent, wherein at least one bioactive agent has a therapeutic or a prophylactic activity for the disease.

33. The method of claim 32, wherein the bioactive agent is selected from the group consisting of a small molecule, a therapeutic agent, an anti-viral agent, a bacteriostatic or anti-bacterial agent, an anti-fungal agent, a cell-targeting ligand, a peptide, a protein, a carbohydrate, a diagnostic agent, and a viral or bacterial protein capable of eliciting a humoral or cellular-based immune response.

34. The method of claim 33, wherein the therapeutic agent is paclitaxel.

35. The method of claim 33, wherein the therapeutic agent comprises an anti-cancer agent and the method further comprises a method of treating a resistance to the anti-cancer agent.

36. The method of claim 32, wherein the administration comprises an administration route selected from the group consisting of parenteral, topical, rectal, oral, inhalation, intranasal, transdermal, and buccal administration.

37. The method of claim 36, wherein the administration route is a topical administration.

38. A method of making a nanocapsule or a nanoemulsion particle comprising a pharmaceutically acceptable liquid oil phase, a surfactant, and optionally a co-surfactant; wherein the liquid oil phase comprises one or more compounds having the structure: 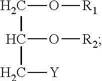
wherein:
Y is selected from the group consisting of H and —O—R₃;
R₁, R₂, and R₃ are each independently selected from the group consisting of 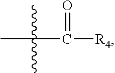
and H; wherein if R₁ is H and R₂ is H, then Y is not H and R₃ is not H;
R₄ is selected from the group consisting of C₁-C₂₅ alkyl, C₁-C₂₅ alkenyl, C₁-C₂₅alkylyl, and 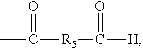
wherein R₅ is —(CH₂)ₓ—, and wherein x is an integer from 1 to 12;
the method comprising admixing the liquid oil phase, the surfactant, and the co-surfactant with an aqueous solvent or a non-aqueous solvent; wherein high pressure mechanical agitation, microfluidization, or heating is not required to produce the nanocapsule or nanoemulsion particle.

39. The method of claim 38, wherein the method comprises heating the liquid oil phase, the surfactant, and the co-surfactant during the admixing with the aqueous solvent or the non-aqueous solvent to produce the nanocapsule or nanoemulsion particle.

40. The method of claim 38, wherein the liquid oil phase, the surfactant, and the co-surfactant are not heated during the admixing with the aqueous solvent or the non-aqueous solvent.
